(19) United States
(12) Patent Application Publication
(10) Pub. No.: US 2024/0319596 A1
(43) Pub. Date: Sep. 26, 2024

(54) RADIATION-SENSITIVE COMPOSITION, RESIST PATTERN FORMATION METHOD, POLYMER, AND COMPOUND

(71) Applicant: JSR CORPORATION, Tokyo (JP)

(72) Inventors: Hiromu MIYATA, Tokyo (JP); Takuhiro TANIGUCHI, Tokyo (JP)

(73) Assignee: JSR CORPORATION, Tokyo (JP)

(21) Appl. No.: 18/574,367

(22) PCT Filed: Jun. 1, 2022
(86) PCT No.: PCT/JP2022/022358

§ 371 Date: Dec. 27, 2023

(30) Foreign Application Priority Data
Jun. 30, 2021 (JP) 2021-109611

(51) Int. Cl. G03F 7/039; C08F 212/08; C08F 220/18; G03F 7/00; G03F 7/004

(52) U.S. Cl. CPC G03F 7/0397; C08F 212/08; C08F 220/18; G03F 7/0002; G03F 7/0045

(57) ABSTRACT

A polymer containing a structural unit (I) represented by formula (1) is included in a radiation-sensitive composition. In formula (1), R² is a single bond, a divalent hydrocarbon group, or the like. R³ is a divalent group represented by formula (2) or formula (3). R⁴ is a divalent organic group. Y⁻ is a monovalent anion which can generate a sulfonic acid group, an imidic acid group, or a methide acid group through exposure to light. M^{a+} is an a-valent cation. a is 1 or 2.

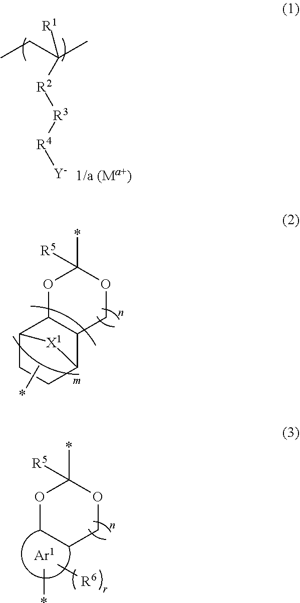

### CROSS-REFERENCE TO RELATED APPLICATIONS

The present application claims the benefit of Japanese Patent Application No. 2021-109611 filed Jun. 30, 2021, the disclosure of which is incorporated herein by reference.

The present disclosure relates to a radiation-sensitive composition, to a resist pattern formation method, to a polymer, and to a compound.

### BACKGROUND ART

In a lithography technique employed in production of various electronic devices including semiconductor devices and liquid crystal devices, a process target formed of a radiation-sensitive composition is irradiated with a far-UV ray (e.g., ArF excimer laser light), an extreme UV (EUV) ray, an electron beam, or the like, to thereby generate acid in a light-exposed part. Through chemical reaction involving the generated acid, difference in dissolution rate with respect to a developer is provided between the exposed part and the unexposed part. Thus, a resist pattern is formed on a substrate.

Structures of such electronic devices have been further miniaturized steeply. Under such circumstances, further fine resist patterns are required in lithography steps. In order to satisfy the requirement, there have been studied improvement in sensitivity and resolution of the radiation-sensitive composition employed in lithographic micro-processing, shape characteristics of a resultant resist pattern, and the like (see, for example, Patent Document 1).

Patent Document 1 discloses incorporation, into a radiation-sensitive composition, of a resin having repeating units which can generate an acid via decomposition through irradiation with active light or radiation.

### PRIOR ART DOCUMENTS

### Patent Documents

Patent Document 1: Japanese Patent Application Laid-Open (kokai) No. 2011-154216

### SUMMARY OF THE INVENTION

### Problems to be Solved by the Invention

In more fine resist patterns, only a slight variation in process conditions (e.g., conditions for exposure and development) readily causes morphological problems, occurrence of failures, and the like which are involved in resist patterns. Thus, the radiation-sensitive composition must have such wide allowance that can accept slight variation in process conditions. That is, there is demand for wide ranges of process conditions which ensure formation of a pattern having no bridge defects and collapses in the resist pattern formation step (hereinafter, such a range may also be referred to as a “process window”).

The disclosure has been made in view of the aforementioned problems. Thus, objects of the disclosure are to provide a radiation-sensitive composition having high sensitivity and a wide process window and to provide a resist pattern formation method.

### Means for Solving the Problems

According to the present disclosure, the following means are provided.

[1] A radiation-sensitive composition including a polymer which contains a structural unit (I) represented by formula (1):

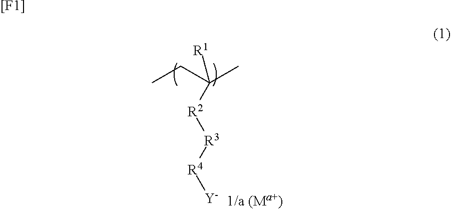

(wherein R¹ represents a hydrogen atom, a fluorine atom, a methyl group, or a trifluoromethyl group; R² represents a single bond, a divalent hydrocarbon group (i.e., a hydrocarbylene group), a divalent group F¹ which is formed by substituting any hydrogen atom of a divalent hydrocarbon group with a monovalent substituent, a divalent group F² including —O—, —CO—, —NH—, —COO—, or —CONH— which intervenes between a C—C bond of a divalent hydrocarbon group or the F¹, *¹—COO—R⁷—, or *¹—CONH—R⁷—; R⁷ represents a single bond, a divalent hydrocarbon group, a divalent group F³ which is formed by substituting any hydrogen atom of a divalent hydrocarbon group with a monovalent substituent, or a divalent group F⁴ including —O—, —CO—, —NH—, —COO—, or —CONH— which intervenes between a C—C bond of a divalent hydrocarbon group or the F³; “*¹” represents a chemical bond to be linked to a carbon atom to which R¹ is bonded; R³ represents a divalent group represented by formula (2) or (3):

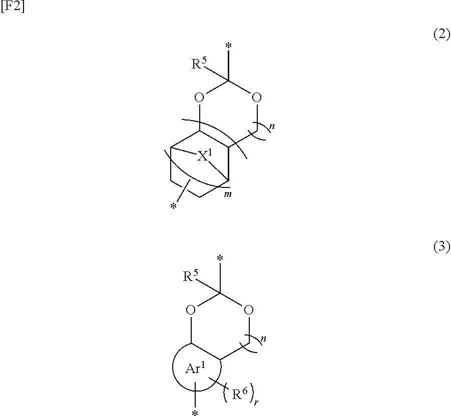

(wherein R⁵ represents a hydrogen atom or a monovalent organic group; X¹ represents —CH₂—, —NH—, —O—, or —S—; Ar¹ represents a ring structure which forms a condensed ring with an oxygen-containing hetero-monocycle in formula (3); R⁶ represents a monovalent substituent; n is 0 or 1; m is 0 or 1; r is an integer of 0 to 2; and “*” represents a chemical bond), wherein, when R² is bonded to an oxygen-containing hetero-monocycle in formula (2) or (3), R⁷ is not a single bond; R⁴ represents a divalent organic group; when R⁴ is bonded to an oxygen-containing hetero-monocycle in formula (2) or (3), R⁴ is bonded to R³ via a carbon atom; Y⁻ represents a monovalent anion which can generate a sulfonate group, an imidic acid group, or a methide acid group through exposure to light; M^{a+} represents an a-valent cation; and a is 1 or 2).

[2] A resist pattern formation method, including a step of forming a resist film on a substrate by use of a radiation-sensitive composition as recited in [1] above, a step of exposing the resist film to light, and a step of developing the light-exposed resist film.

[3] A polymer containing a structural unit represented by formula (1).

[4] A compound represented by formula (4):

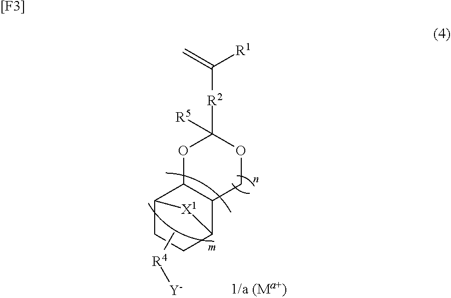

(wherein R¹ represents a hydrogen atom, a fluorine atom, a methyl group, or a trifluoromethyl group; R² represents a single bond, a divalent hydrocarbon group, a divalent group F¹ which is formed by substituting any hydrogen atom of a divalent hydrocarbon group with a monovalent substituent, a divalent group F² including —O—, —CO—, —NH—, —COO—, or —CONH— which intervenes between a C—C bond of a divalent hydrocarbon group or the F¹, *¹—COO—R⁷—, or *¹—CONH—R⁷—; R⁷ represents a divalent hydrocarbon group, a divalent group F³ which is formed by substituting any hydrogen atom of a divalent hydrocarbon group with a monovalent substituent, or a divalent group F⁴ including —O—, —CO—, —NH—, —COO—, or —CONH— which intervenes between a C—C bond of a divalent hydrocarbon group or the F³; “*¹” represents a chemical bond to be linked to a carbon atom to which R¹ is bonded; R⁴ represents a divalent organic group; R⁵ represents a hydrogen atom or a monovalent organic group; X¹ represents —CH₂—, —NH—, —O—, or —S—; Y⁻ represents a monovalent anion which can generate a sulfonate group, an imidic acid group, or a methide acid group through exposure to light; M^{a+} represents an a-valent cation; n is 0 or 1; m is 0 or 1; and a is 1 or 2).

[5] A compound represented by formula (5):

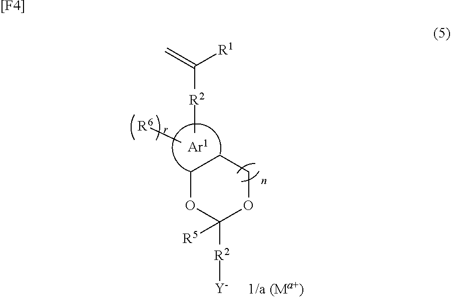

(wherein R¹ represents a hydrogen atom, a fluorine atom, a methyl group, or a trifluoromethyl group; R² represents a single bond, a divalent hydrocarbon group, a divalent group F¹ which is formed by substituting any hydrogen atom of a divalent hydrocarbon group with a monovalent substituent, a divalent group F² including —O—, —CO—, —NH—, —COO—, or —CONH— which intervenes between a C—C bond of a divalent hydrocarbon group or the F¹, *¹—COO—R⁷—, or *¹—CONH—R⁷—; R⁷ represents a single bond, a divalent hydrocarbon group, a divalent group F³ which is formed by substituting any hydrogen atom of a divalent hydrocarbon group with a monovalent substituent, or a divalent group F⁴ including —O—, —CO—, —NH—, —COO—, or —CONH— which intervenes between a C—C bond of a divalent hydrocarbon group or the F³; “*i” represents a chemical bond to be linked to a carbon atom to which R¹ is bonded; Ar¹ represents a ring structure which forms a condensed ring with an oxygen-containing hetero-monocycle in formula (5); R⁴ represents a divalent organic group, but R⁴ is bonded to an oxygen-containing hetero-monocycle at a carbon atom; R⁵ represents a hydrogen atom or a monovalent organic group; R⁶ represents a monovalent substituent; n is 0 or 1; r is an integer of 0 to 2; Y⁻ represents a monovalent anion which can generate a sulfonate group, an imidic acid group, or a methide acid group through exposure to light; M^{a+} represents an a-valent cation; and a is 1 or 2).

### Advantageous Effects of the Invention

Since the radiation-sensitive composition of the present disclosure has high sensitivity, a more favorable resist pattern can be formed by a small light exposure dose. In addition, the radiation-sensitive composition of the present disclosure has a wide process window. Thus, a resist pattern can be formed while an undesired effect due to variation in process conditions can be suppressed.

### MODES FOR CARRYING OUT THE INVENTION

### <Radiation-Sensitive Composition>

The radiation-sensitive composition of the present disclosure (hereinafter may also be referred to simply as “the present composition”) contains polymer [A]. The present composition may further contain, as a suitable component, at least one member of an acid generator [B], acid diffusion control agent [C], a solvent [D], an acid-releasable group-containing polymer [E], and a high-fluorine content polymer [F]. These components will next be described in detail.

As used herein, the term “hydrocarbon group” encompasses a chain hydrocarbon group, an alicyclic hydrocarbon group, and an aromatic hydrocarbon group. The term “chain hydrocarbon group” refers to a linear-chain hydrocarbon group or a branched hydrocarbon group including one which is composed of only a chain structure and no ring structure. However, the chain hydrocarbon group may be saturated or unsaturated. The term “alicyclic hydrocarbon group” refers to a hydrocarbon group which contains only an alicyclic hydrocarbon moiety as a ring structure and contains no aromatic ring structure. However, the alicyclic hydrocarbon group is not necessarily formed only of an alicyclic hydrocarbon moiety and may contain a chain structure as a partial structure. The term “aromatic hydrocarbon group” refers to a hydrocarbon group which contains an aromatic ring structure as a ring structure. However, the aromatic hydrocarbon group is not necessarily formed only of an aromatic ring structure and may contain a chain structure or an alicyclic hydrocarbon moiety as a partial structure. The term “organic group” refers to an atomic group formed by removing any hydrogen atom from a carbon-containing compound (i.e., an organic compound). The expression “(meth)acrylic” encompasses “acrylic” and “methacrylic.” The expression “(meth)acryloyl” encompasses “acryloyl” and “methacryloyl.” The expression “(meth)acrylate” encompasses “acrylate” and “methacrylate.”

### <[A] Polymer>

Polymer [A] is a polymer containing a structural unit (I) represented by formula (1).

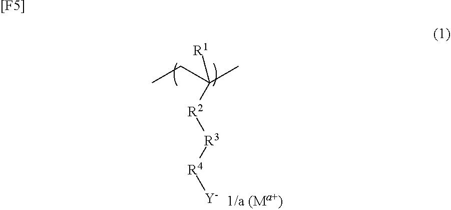

(In formula (1), R¹ represents a hydrogen atom, a fluorine atom, a methyl group, or a trifluoromethyl group; R² represents a single bond, a divalent hydrocarbon group, a divalent group F¹ which is formed by substituting any hydrogen atom of a divalent hydrocarbon group with a monovalent substituent, a divalent group F² including —O—, —CO—, —NH—, —COO—, or —CONH— which intervenes between a C—C bond of a divalent hydrocarbon group or F¹, *¹—COO—R⁷—, or *¹—CONH—R⁷—; R⁷ represents a single bond, a divalent hydrocarbon group, a divalent group F³ which is formed by substituting any hydrogen atom of a divalent hydrocarbon group with a monovalent substituent, or a divalent group F⁴ including —O—, —CO—, —NH—, —COO—, or —CONH— which intervenes between a C—C bond of a divalent hydrocarbon group or the F³; “*¹” represents a chemical bond to be linked to a carbon atom to which R¹ is bonded; R³ represents a divalent group represented by formula (2) or (3), wherein, when R² is bonded to an oxygen-containing hetero-monocycle in formula (2) or (3), R⁷ is not a single bond; R⁴ represents a divalent organic group; when R⁴ is bonded to an oxygen-containing hetero-monocycle in formula (2) or (3), R⁴ is bonded to R³ via a carbon atom; Y⁻ represents a monovalent anion which can generate a sulfonate group, an imidic acid group, or a methide acid group through exposure to light; M^{a+} represents an a-valent cation; and a is 1 or 2)

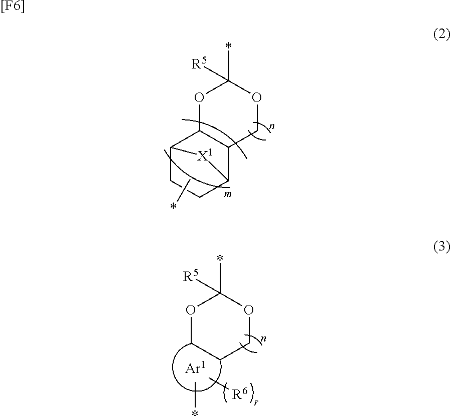

(In formulas (2) and (3), R⁵ represents a hydrogen atom or a monovalent organic group; X¹ represents —CH₂—, —NH—, —O—, or —S—; Ar¹ represents a ring structure which forms a condensed ring with an oxygen-containing hetero-monocycle in formula (3); R⁶ represents a monovalent substituent; n is 0 or 1; m is 0 or 1; r is an integer of 0 to 2; and “*” represents a chemical bond.)

In formula (1), examples of the divalent hydrocarbon group represented by R² or R⁷ include a C1 to C20 divalent chain hydrocarbon group, a C3 to C20 divalent alicyclic hydrocarbon group, and a C6 to C20 divalent aromatic hydrocarbon group.

Examples of the C1 to C20 divalent chain hydrocarbon group include alkanediyls such as methylene, ethylene, n-propylene, and isopropylene; alkenediyls such as ethylenediyl, propylenediyl, and butylenediyl; and alkynediyls such as ethynediyl, propynediyl, and butynediyl. Among them, the divalent chain hydrocarbon group represented by R² is preferably a C1 to C20 alkanediyl group, more preferably a C1 to C5 alkanediyl group, still more preferably a C1 to C3 alkanediyl. The divalent chain hydrocarbon group represented by R⁷ is preferably a C1 to C20 alkanediyl group, more preferably a C1 to C10 alkanediyl group, still more preferably a C1 to C4 alkanediyl group.

Examples of the C3 to C20 divalent alicyclic hydrocarbon group include divalent monocyclic alicyclic saturated hydrocarbon groups such as cyclopentylene and cyclohexylene; divalent monocyclic alicyclic unsaturated hydrocarbon groups such as cyclopentenediyl and cyclohexenediyl; divalent polycyclic alicyclic saturated hydrocarbon groups such as norbornanediyl and adamantanediyl; and divalent polycyclic alicyclic unsaturated hydrocarbon groups such as norbornenediyl. Among them, the divalent alicyclic hydrocarbon group represented by R² or R⁷ is preferably a divalent monocyclic alicyclic saturated hydrocarbon group or a divalent polycyclic alicyclic saturated hydrocarbon group, more preferably cyclopentylene, cyclohexylene, norbornanediyl, or adamantanediyl.

Examples of the C6 to C20 divalent aromatic hydrocarbon group include arylene groups such as phenylene, tolylene, xylylene, trimethylphenylene, naphthylene, and methylnaphthylene; and aralkylene groups such as -A¹⁰-R¹⁰— (wherein A¹⁰ represents a phenylene group or a naphthylene group; and R¹⁰ represents a C1 to C3 alkanediyl group).

Among the above groups, the divalent hydrocarbon group represented by R² is preferably a divalent aromatic hydrocarbon group, more preferably an arylene group, still more preferably a phenylene group or a naphthylene group. The divalent hydrocarbon group represented by R⁷ is preferably a divalent chain hydrocarbon group, more preferably a C1 to C20 alkanediyl group, still more preferably a C1 to C4 alkanediyl group.

When R² or R⁷ is a divalent group (F¹,F³) which is formed by substituting any hydrogen atom of a divalent hydrocarbon group with a monovalent substituent, examples of the substituent which can change a hydrogen atom include a halogen atom (e.g., fluorine atom, chlorine atom, bromine atom, or iodine atom), a hydroxy group, a cyano group, a nitro group, and a C1 to C3 alkoxy group. Specific examples of the divalent hydrocarbon group include those exemplified in relation to the divalent hydrocarbon group represented by R² or R⁷. In groups F¹ and F³, no particular limitation is imposed on the number of hydrogen atom(s) to be substituted, and the number is, for example, 1 to 6.

Alternatively, one or both of R² and R⁷ may be a divalent group (F², F⁴) including —O—, —CO—, —NH—, —COO—, or —CONH— which intervenes between a C—C bond of a divalent hydrocarbon group, group F¹, or group F³. Specific examples of the divalent hydrocarbon group include those exemplified in relation to the divalent hydrocarbon group represented by R² or R⁷. Notably, when R² is bonded to an oxygen-containing hetero-monocycle in formula (2) or (3), R⁷ is not a single bond.

R³ is a divalent group represented by formula (2) or (3). In formulas (2) and (3), the monovalent organic group represented by R⁵ is preferably a C1 to C10 alkyl group, more preferably a C1 to C3 alkyl group. In formula (2), X¹ is preferably —CH₂—, —NH—, or —O—, more preferably —CH₂—.

In formula (3), the ring structure represented by Ar¹ is preferably an aromatic ring structure, for example, a benzene ring structure or a naphthalene structure. Examples of the monovalent substituent represented by R⁶ include a C1 to C3 alkyl group, a C1 to C3 alkoxy group, a hydroxy group, and a halogen atom.

Examples of the divalent organic group represented by R⁴ include a divalent hydrocarbon group and a divalent group having a fluorine atom. When the divalent organic group represented by R⁴ is a divalent hydrocarbon group, specific examples include groups as exemplified in the description of the divalent hydrocarbon group represented by R² or R⁷. Among them, the divalent hydrocarbon group represented by R⁴ is preferably an aromatic hydrocarbon group, from the viewpoint of easy synthesis of a monomer which provides a structural unit (I).

When the divalent organic group represented by R⁴ is a divalent group having a fluorine atom, R⁴ is preferably a group represented by formula (6).

*²—R⁸—R^{f1}- (6)

(In formula (6), R⁸ is a single bond or a divalent linkage group; R^{f1} represents a C1 to C10 fluorinated alkanediyl group; and “*²” represents a chemical bond to be bonded to R³ in formula (1), wherein, when R⁴ is bonded to an oxygen-containing hetero-monocycle in formula (2) or (3), R⁸ is bonded to R³ via a carbon atom.)

When R⁴ is not directly bonded to an oxygen-containing hetero-monocycle in formula (2) or (3) (i.e., R⁴ is bonded to a bridge ring in formula (2) or to Ar¹ in formula (3)), specific examples of the divalent linkage group represented by R⁸ in formula (6) include a C1 to C6 alkanediyl group and a divalent group formed by substituting any methylene group of a C1 to C6 alkanediyl group with —O— or —COO—. When R⁴ is directly bonded to an oxygen-containing hetero-monocycle in formula (2) or (3), the divalent linkage group represented by R⁸ is preferably a C1 to C6 alkanediyl group.

The fluorinated alkanediyl group represented by R^{f1} is preferably a group represented by the following formula (f-1).

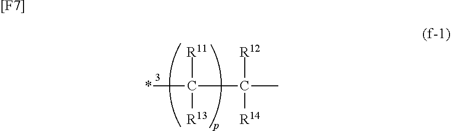

(In formula (f-1), each of R¹¹ and R¹² represents a hydrogen atom, a fluorine atom, or a C1 to C3 perfluoroalkyl group. Each of R¹³ and R¹⁴ represents a fluorine atom or a C1 to C3 perfluoroalkyl group; p is an integer of 0 to 3; and “*³” represents a chemical bond bonded to R⁸ in formula (6).)

In formula (f-1), each of R¹³ and R¹⁴ is preferably a fluorine atom or a trifluoromethyl group. R¹² is preferably a fluorine atom or a C1 to C3 perfluoroalkyl group, more preferably a fluorine atom or a trifluoromethyl group. R¹¹ is preferably a hydrogen atom, a fluorine atom, or a C1 to C3 perfluoroalkyl group, more preferably a hydrogen atom, a fluorine atom, a trifluoromethyl group.

Specific examples of the fluorinated alkanediyl group represented by R^{f1} include a fluoromethanediyl group, a difluoromethanediyl group, a 1,2-difluoroethane-1,2-diyl group, a 1,1,2-trifluoroethane-1,2-diyl, a 1,1,2,2-tetrafluoroethane-1,2-diyl group, and a 1,1,3,3,3-pentafluoropropane-1,2-diyl group.

From the viewpoints of enhancing the sensitivity of the present composition and easy synthesis of a monomer which provides a structural unit (I), R⁴ is preferably a divalent group having a fluorine atom, in the case where Y⁻ is a monovalent anion which can generate a sulfonate group or a methide acid group through exposure to light. Also, when Y⁻ is a monovalent anion which can generate an imidic acid group, R⁴ is preferably a divalent hydrocarbon group or a divalent group having a fluorine atom.

Y⁻ is a monovalent anion which can generate a sulfonate group, an imidic acid group, or a methide acid group through exposure to light. Specifically, Y⁻ is preferably a group represented by formula (Y-1), a group represented by formula (Y-2), or a group represented by formula (Y-3).

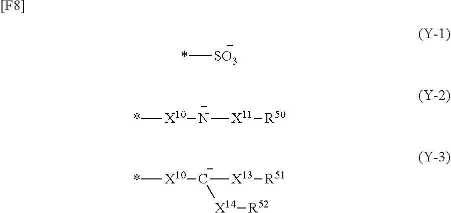

(In formulas (Y-1) to (Y-3), each of X¹⁰, X¹¹, X¹², X¹³, and X¹⁴ represents —CO— or —SO₂—; each of R⁵⁰, R⁵¹ and R⁵² represents a monovalent hydrocarbon group (i.e., a hydrocarbyl group) or a monovalent group formed by substituting any hydrogen atom of a monovalent hydrocarbon group with a substituent; and “*” represents a chemical bond.)

In formulas (Y-1) to (Y-3), examples of monovalent hydrocarbon groups represented by R⁵⁰, R⁵¹, or R⁵² include a C1 to C20 monovalent chain hydrocarbon group, a C3 to C20 monovalent alicyclic hydrocarbon group, and a C6 to C20 monovalent aromatic hydrocarbon group.

Examples of the C1 to C20 monovalent chain hydrocarbon group include alkyl groups such as methyl, ethyl, n-propyl, and i-propyl; alkenyl groups such as ethenyl, propenyl, and butenyl; and alkynyl groups such as ethynyl, propynyl, and butynyl. Of these, an alkyl group is preferred, with a C1 to C4 alkyl group being more preferred, as the C1 to C20 monovalent chain hydrocarbon group represented by R⁵⁰, R⁵¹, or R⁵².

Examples of the C3 to C20 monovalent alicyclic hydrocarbon group include monovalent monocyclic alicyclic saturated hydrocarbon groups such as cyclopentyl and cyclohexyl; monovalent monocyclic alicyclic unsaturated hydrocarbon groups such as cyclopentenyl and cyclohexenyl; monovalent polycyclic alicyclic saturated hydrocarbon groups such as norbornyl, adamantyl, and tricyclodecyl; and monovalent polycyclic alicyclic unsaturated hydrocarbon groups such as norbornenyl and tricyclodecenyl.

Examples of the C6 to C20 monovalent aromatic hydrocarbon group include aryl groups such as phenyl, tolyl, xylyl, mesityl, naphthyl, methylnaphthyl, anthryl, and methyl anthryl; and aralkyl groups such as benzyl, phenethyl, naphtylmethyl, and anthrylmethyl.

Among the aforementioned groups, each of R⁵⁰, R⁵¹, and R⁵² is preferably a group formed by substituting any hydrogen atom of the monovalent hydrocarbon group with a fluorine atom, is more preferably a C1 to C10 fluoroalkyl group.

Preferably, the structural unit (I) is at least one species selected from the group consisting of, in particular, structural units derived from a compound represented by formula (4) (hereinafter may also be referred to as “compound (a1)”) and those derived from a compound represented by formula (5) (hereinafter may also be referred to as “compound (a2)”).

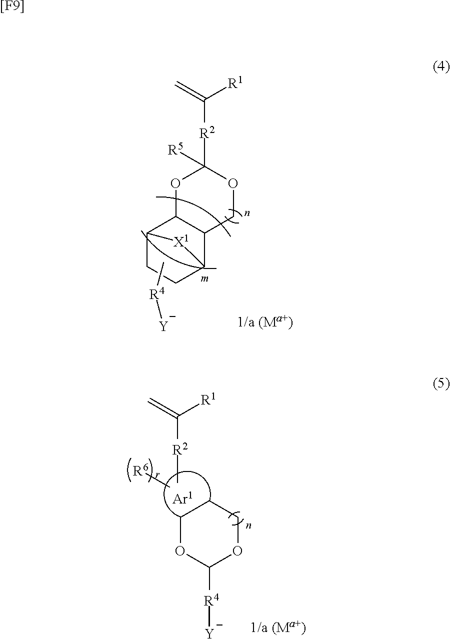

(In formulas (4) and (5), R¹, R², R⁴, R⁵, R⁶, Y⁻, M^{a+}, X¹, Ar¹, a, n, m, and r have the same meanings as defined in formulas (1) to (3).)

From the viewpoint of copolymerization performance, the monomer forming the structural unit (I), at least one species selected from the group consisting of a styrenic monomer and a (meth)acrylic monomer is preferred. Specific examples of preferred monomers each forming the structural unit (I) include a compound represented by formula (4-1A), a compound represented by formula (4-2A), and a compound represented by formula (5-1A).

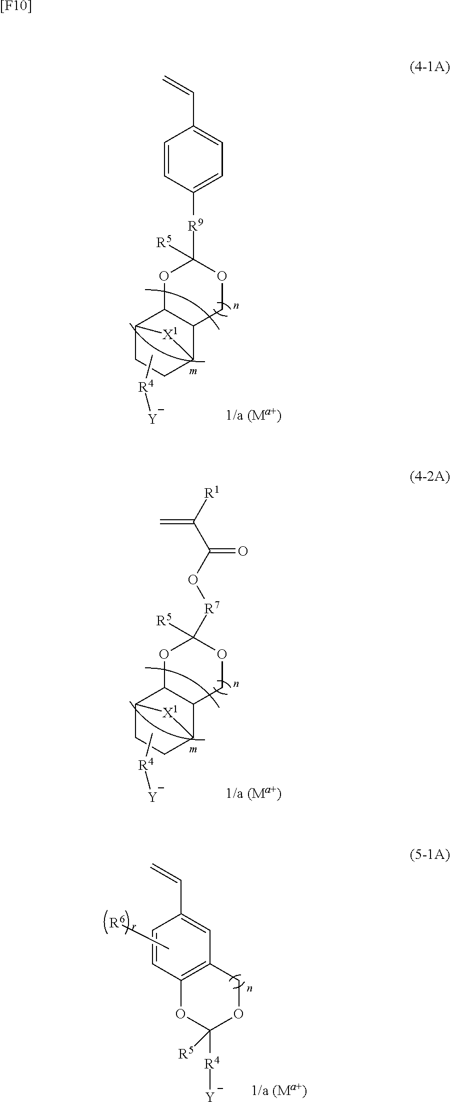

(In formulas (4-1A), (4-2A), and (5-1A), R⁹ is a single bond, a divalent hydrocarbon group, a divalent group F⁵ which is formed by substituting any hydrogen atom of a divalent hydrocarbon group with a monovalent substituent, or a divalent group F⁶ including —O—, —CO—, —NH—, —COO—, or —CONH— which intervenes between a C—C bond of a divalent hydrocarbon group or group F⁵; and R¹, R⁴, R⁵, R⁶, R⁷, Y⁻, M^{a+}, X¹, a, n, m, and r have the same meanings as defined in formulas (1) to (3))

In formula (4-1A), examples of the divalent hydrocarbon group represented by R⁹ include a C1 to C14 divalent chain hydrocarbon group, a C3 to C14 divalent alicyclic hydrocarbon group, and a C6 to C14 divalent aromatic hydrocarbon group. Among them, the divalent hydrocarbon group represented by R⁹ is preferably a divalent chain hydrocarbon group, more preferably a C1 to C5 alkanediyl group.

In the case where R⁹ is a divalent group F⁵ which is formed by substituting any hydrogen atom of a divalent hydrocarbon group with a monovalent substituent, examples of the substituent replacing the hydrogen atom include a halogen atom, a hydroxy group, a cyano group, a nitro group, and a C1 to C3 alkoxy group. Also, R⁹ may be a divalent group (F⁶) including —O—, —CO—, —NH—, —COO—, or —CONH— which intervenes between a C—C bond of a divalent hydrocarbon group or group F⁵.

Specific examples of the monomer forming the structural unit (I) include compounds represented by formula (4); e.g., compounds represented formulas (4-1) to (4-12), respectively, and compounds represented by formula (5); e.g., compounds represented formulas (5-1) to (5-4), respectively.

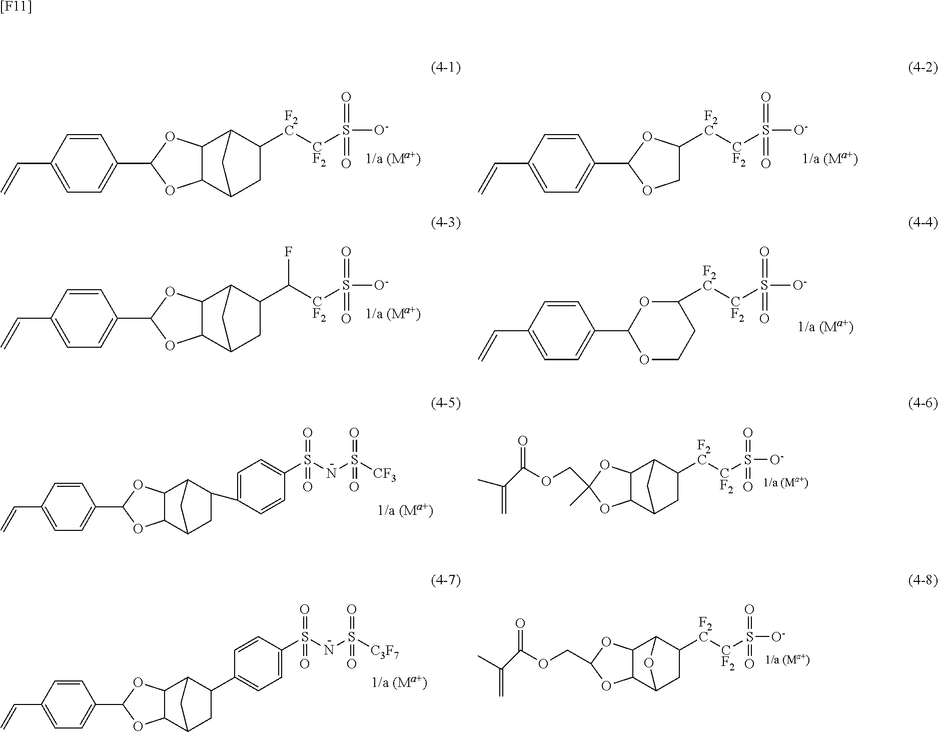

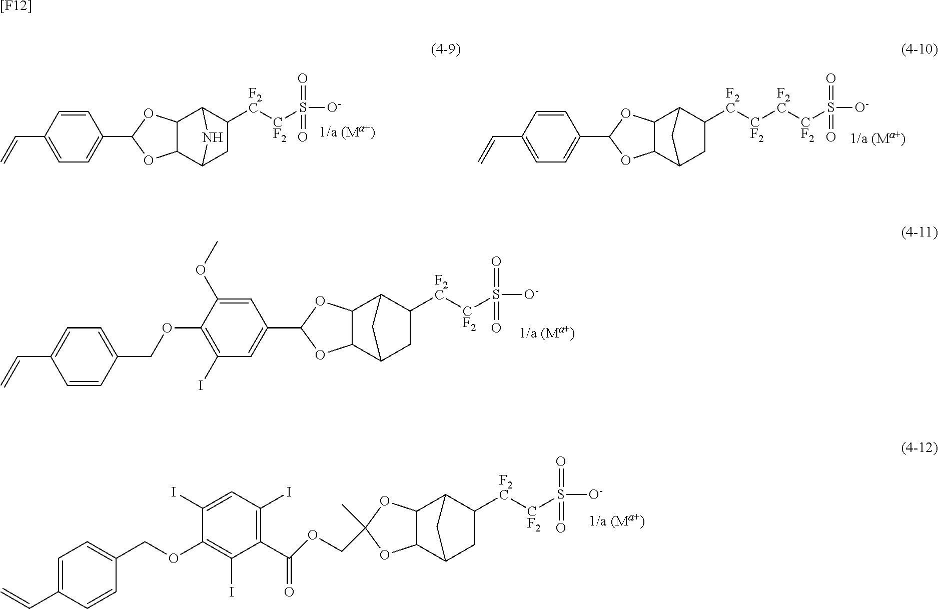

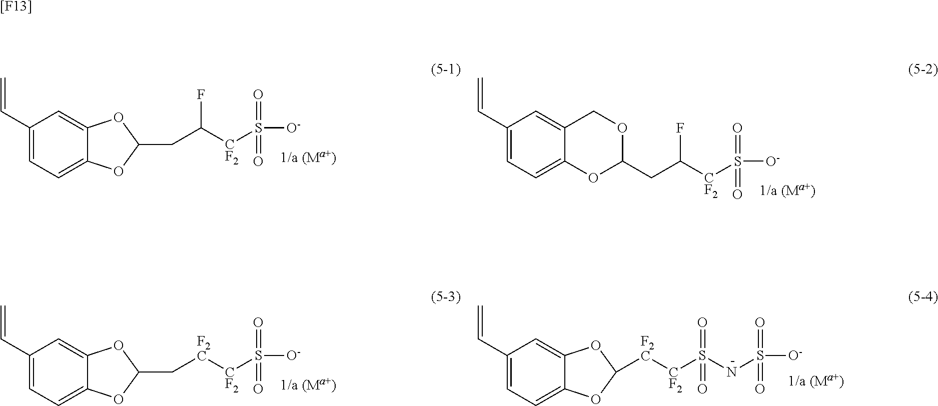

(In formulas (4-1) to (4-12) and (5-1) to (5-4), M^{a+} represents an a-valent cation, and a is 1 or 2)

### Cation in Formula (1)

M^{a+} in formula (1) is preferably an organic cation, particularly preferably a radiation-sensitive onium cation. When M^{a+} is a radiation-sensitive onium cation, each of compounds (a1) and (a2) is an onium salt.

No particular limitation is imposed on the structure of M^{a+}. From the viewpoint of achieving favorable lithographic characteristics of the present composition, a sulfonium cation, an iodonium cation, or an ammonium cation is preferred, with a sulfonium cation and an iodonium cation being more preferred.

In the case where “a” in formula (1) is 1, specific examples of M^{a+} include a cation represented by formula (7), a cation represented by formula (8), and a cation represented by formula (9).

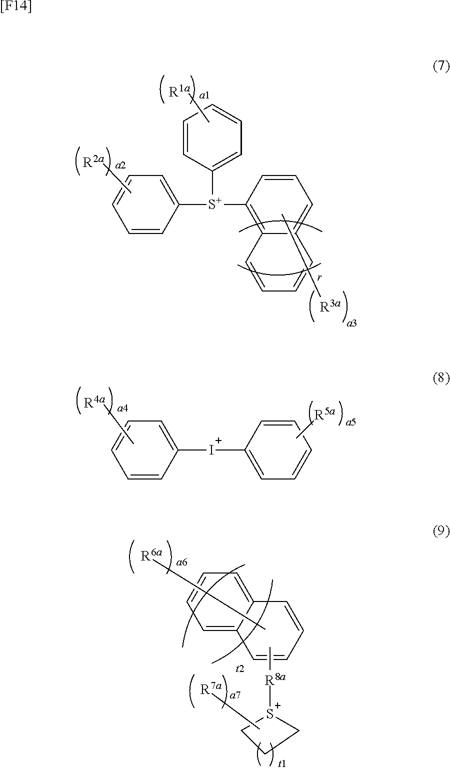

(In formula (7), each of R^{1a} and R^{2a} represents a monovalent substituent, or a single bond or a divalent group linked to a ring formed by combining R^{1a} and R^{2a}; R^{3a} represents a monovalent substituent; each of a1 and a2 is an integer of 0 to 5; a3 is an integer of 0 to (2×r+5); and r is 0 or 1).

In formula (8), each of R^{4a} and R^{5a} represents a monovalent substituent. Each of a4 and a5 is an integer of 0 to 5.

In formula (9), a6 is an integer of 0 to 7. When a6 is 1, R^{6a} is a C1 to C20 monovalent organic group, a hydroxy group, a nitro group, or a halogen group. When a6 is 2 or greater, a plurality of R^{6a}s, which are identical to or different from one another, represents a C1 to C20 monovalent organic group, a hydroxy group, a nitro group, or a halogen group, or a 4- to 20-membered ring structure formed with a carbon atom to which a linked moiety two of a plurality of R^{6a}s are bonded. The number a7 is an integer of 0 to 6. When a7 is 1, R^{7a} is a C1 to C20 monovalent organic group, a hydroxy group, a nitro group, or a halogen group. When a7 is 2 or greater, a plurality of R^{7a}s, which are identical to or different from one another, represents a C1 to C20 monovalent organic group, a hydroxy group, a nitro group, or a halogen group, or a 3- to 20-membered ring structure formed with a carbon atom to which a linked moiety formed of two of plurality of R^{7a}s are bonded. The number t1 is an integer of 0 to 3. R^{8a} represents a single bond or a C1 to C20 divalent organic group. The number t2 is 0 or 1.)

In formulas (7) and (8), examples of the monovalent substituent represented by R^{1a}, R^{2a}, R^{3a}, R^{4a}, or R^{5a} (hereinafter denoted by “R^{1a} to R^{5a}”) include a fluorine atom, a chlorine atom, a bromine atom, an iodine atom, a substituted or unsubstituted alkyl group, a substituted or unsubstituted alkoxy group, a substituted or unsubstituted cycloalkyl group, a substituted or unsubstituted cycloalkyloxy group, an ester group, an alkylsulfonyl group, a cycloalkylsulfonyl group, a hydroxy group, a carboxy group, a cyano group, and a nitro group.

The alkyl group represented by any of R^{1a} to R^{5a} may be linear-chain or branched. The alkyl group is preferably a C1 to C10, and examples thereof include methyl, ethyl, n-propyl, i-propyl, n-butyl, i-butyl, sec-butyl, t-butyl, n-pentyl, and neopentyl. Of these, the alkyl group represented by any of R^{1a} to R^{5a} is preferably a C1 to C5, with methyl, ethyl, n-butyl, and t-butyl being more preferred. When any of R^{1a} to R^{5a} is a substituted alkyl group, examples of the substituent include a fluorine atom, a chlorine atom, a bromine atom, an iodine atom, a hydroxy group, a carboxy group, a cyano group, a nitro group, and a C1 to C5 alkoxy group.

When any of R^{1a} to R^{5a} is a substituted or unsubstituted alkoxy group, specific examples thereof include a group in which any of the above-exemplified substituted or unsubstituted alkoxy groups is attached to the alkyl moiety forming the alkoxy group. The alkoxy group is particularly preferably methoxy, ethoxy, n-propoxy, or n-butoxy.

The cycloalkyl group represented by any of R^{1a} to R^{5a} may be monocyclic or polycyclic. Examples of the monocyclic cycloalkyl group include cyclopropyl, cyclobutyl, cyclopentyl, cyclohexyl, and cyclooctyl. Examples of the polycyclic cycloalkyl group include norbornyl, adamantyl, tricyclodecyl, and tetracyclododecyl. When cycloalkyl group represented by any of R^{1a} to R^{5a} has a substituent, examples of the substituent include a fluorine atom, a chlorine atom, a bromine atom, an iodine atom, a hydroxy group, a carboxy group, a cyano group, a nitro group, and a C1 to C5 alkoxy group.

When any of R^{1a} to R^{5a} is a substituted or unsubstituted cycloalkyloxy group, specific examples thereof include a group in which any of the above-exemplified substituted or unsubstituted cycloalkyl groups is attached to the cycloalkyl moiety forming the cycloalkyloxygroup. The cycloalkyloxy group represented by any of R^{1a} to R^{5a} is particularly preferably a cyclopentyloxy group or a cyclohexyloxy group.

When any of R^{1a} to R^{5a} is an ester group (—COOR), specific examples of the hydrocarbon moiety (R) of the ester group include the above-exemplified, substituted or unsubstituted alkyl groups and substituted or unsubstituted cycloalkyl groups. When any of R^{1a} to R^{5a} is an ester group, R^{1a} to R^{5a} are preferably methoxycarbonyl, ethoxycarbonyl, or n-butoxycarbonyl.

When any of R^{1a} to R^{5a} is an alkylsulfonyl group, examples of the alkyl moiety forming the alkylsulfonium group include the above-mentioned substituted or unsubstituted alkyl groups. When any of R^{1a} to R^{5a} is a cycloalkylsulfonyl group, examples of the alkyl moiety forming the cycloalkylsulfonium group include the aforementioned, substituted or unsubstituted cycloalkyl groups.

When R^{1a} and R^{2a} represent a divalent group linked to a ring formed by combining R^{1a} and R^{2a}, examples of the divalent group include —COO—, —OCO—, —CO—, —O—, —SO—, —SO₂—, —S—, a C1 to C3 alkanediyl group, a C2 or C3 alkenediyl group, and a group in which includes —O—, —S—, —COO—, —OCO—, —CO—, —SO—, or —SO₂— intervening between the C—C bond of an ethylene group. Among them, when R^{1a} and R^{2a} are a single bond or a divalent group linked to a ring formed by combining R^{1a} and R^{2a}, R^{1a} and R^{2a} are preferably a single bond linking ring structures or form —O— or —S—.

The number “a1” is preferably an integer of 0 to 2, more preferably 1 or 2 wherein at least one R^{1a} is a fluorine atom or a trifluoromethyl group. The number “a2” is preferably an integer of 0 to 2, more preferably 1 or 2 wherein at least one R^{2a} is a fluorine atom or a trifluoromethyl group. The number “a3” is preferably an integer of 0 to 2, more preferably 1 or 2 wherein at least one R^{3a} is a fluorine atom or a trifluoromethyl group. Particularly, all of a1, a2, and a3 are independently and preferably an integer of 0 to 2. More preferably, all of a1, a2, and a3 are independently 1 or 2, wherein at least one R^{1a}, at least one R^{2a}, or at least one R^{3a} is a fluorine atom or a trifluoromethyl group.

In formula (9), each of R^{6a} and R^{7a} is preferably a substituted or unsubstituted C1 to C20 monovalent hydrocarbon group, —OR^{k}, —COOR^{k}, —O—CO—R^{k}, —O—R^{kk}—COOR^{k}, —R^{kk}—CO—R^{k}, —OSO₂—R^{k}, or —SO₂—R^{k}. R^{k} represents a C1 to C10 monovalent hydrocarbon group. R^{kk} represents a single bond or a C1 to C10 divalent hydrocarbon group. Examples of the C1 to C20 monovalent hydrocarbon group represented by R^{6a} or R^{7a} include the same groups as exemplified in relation to monovalent hydrocarbon groups represented by R⁵⁰, R⁵¹, or R⁵² in formulas (Y-1) to (Y-3). In R^{6a} and R^{7a}, examples of the substituent which replaces a hydrogen atom of a hydrocarbon group include the same groups as exemplified in relation to substituents included in the aforementioned R^{3a}.

Examples of the divalent organic group represented by R^{8a} include a group formed by removing one hydrogen atom from a C1 to C20 monovalent organic group exemplified in relation to R^{6a} and R^{7a}.

Among the aforementioned examples, each of R^{6a} and R^{7a} is preferably an unsubstituted, linear-chain or branched monovalent alkyl group or monovalent fluoroalkyl group, an unsubstituted monovalent aromatic hydrocarbon group, —OSO₂—R^{k}, or —SO₂—R^{k}. The number “a6” is preferably an integer of 0 to 2, more preferably 0 or 1, still more preferably 0. The number “a7” is preferably an integer of 0 to 2, more preferably 0 or 1, still more preferably 0. The number “t2” is preferably 0. The number “t1” is preferably 2 or 3, more preferably 2.

When the number “a” in formula (1) is 1, M^{a+} is preferably a sulfonium cation or an iodonium cation, more preferably an cation represented by formula (7) or (9), still more preferably a cation represented by formula (7).

When the number “a” in formula (1) is 2, M^{a+} is preferably a sulfonium cation. Specific examples of the sulfonium cation include cations represented by formula (10).

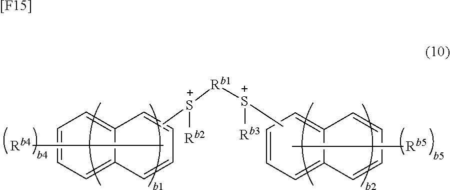

(In formula (10), R^{b1} represents a single bond or a C1 to C20 divalent organic group; each of R^{b2} and R^{b3} represents a C1 to C20 monovalent organic group or a 4- to 20-membered ring structure formed with S⁺—R^{b1}—S⁺ to which a linked moiety of R^{b2} and R^{b3} is bonded; the number “b4” is an integer of 0 to 9; when b4 is 1, R^{b4} is a C1 to C20 monovalent organic group, a hydroxy group, a nitro group, or a halogen atom; when b4 is 2 or greater, a plurality of R^{b4}s, which are identical to or different from one another, represent a C1 to C20 monovalent organic group, a hydroxy group, a nitro group, or a halogen atom, or a 4- to 20-membered ring structure formed with a carbon atom to which a linked moiety formed of a plurality of R^{b4}s is bonded; the number “b5” is an integer of 0 to 9; when b5 is 1, R^{b5} is a C1 to C20 monovalent organic group, a hydroxy group, a nitro group, or a halogen atom; when b5 is 2 or greater, a plurality of R^{b5}s, which are identical to or different from one another, represent a C1 to C20 monovalent organic group, a hydroxy group, a nitro group, or a halogen atom, or a 4- to 20-membered ring structure formed with a carbon atom to which a linked moiety formed of a plurality of R^{b5}s are bonded; b1 is an integer of 0 to 2; and b2 is an integer of 0 to 2.)

No particular limitation is imposed on M^{a+}, and specific examples thereof include cations represented by the following formulas.

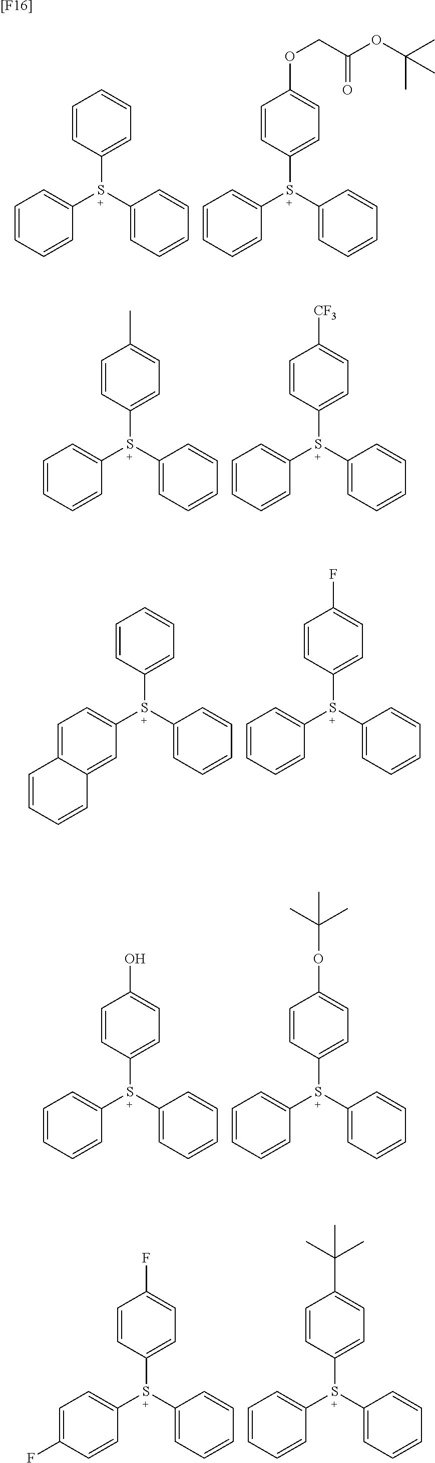

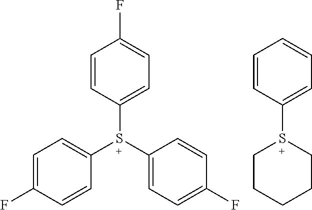

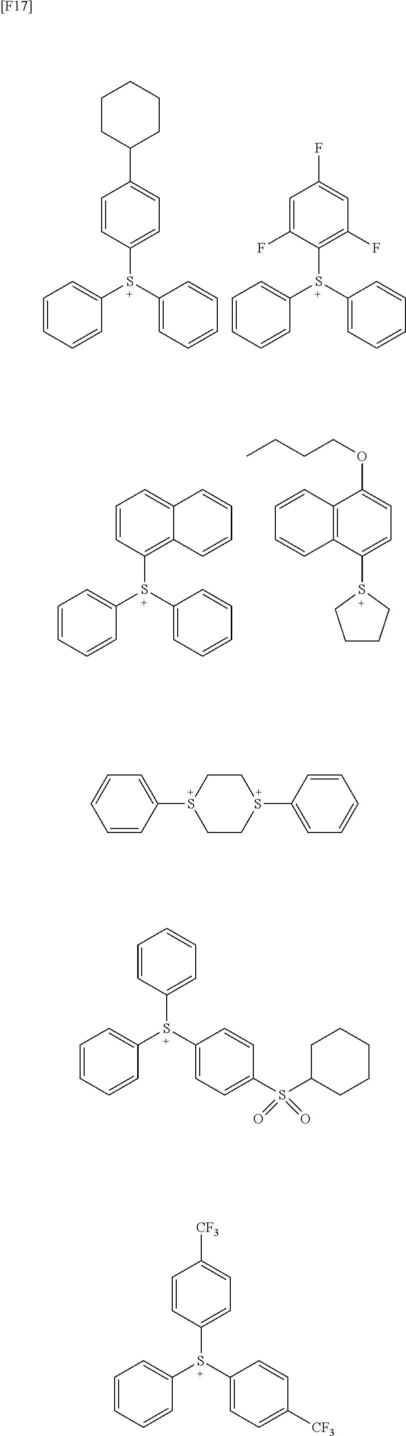

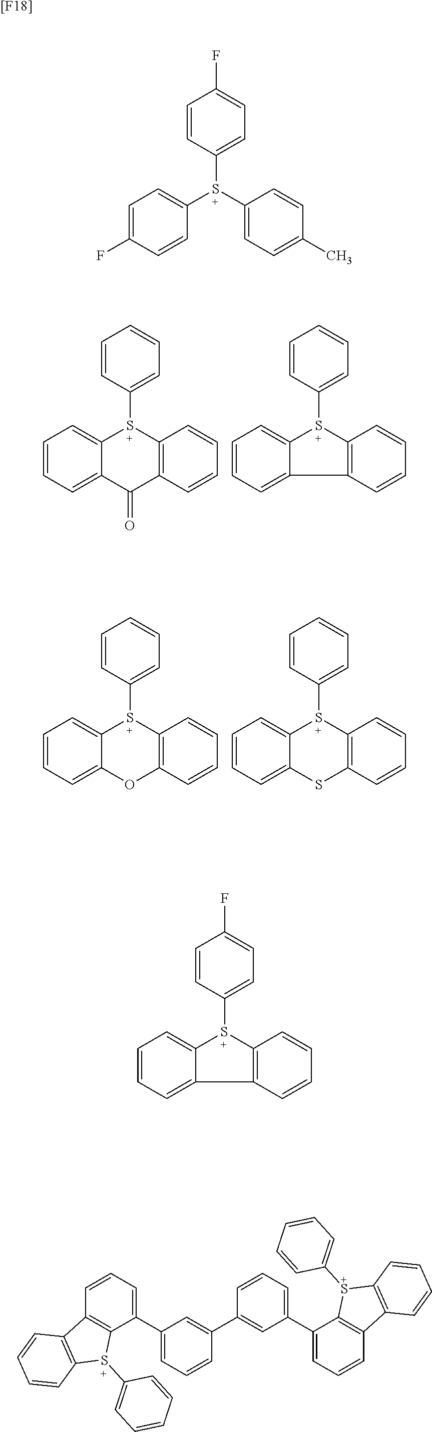

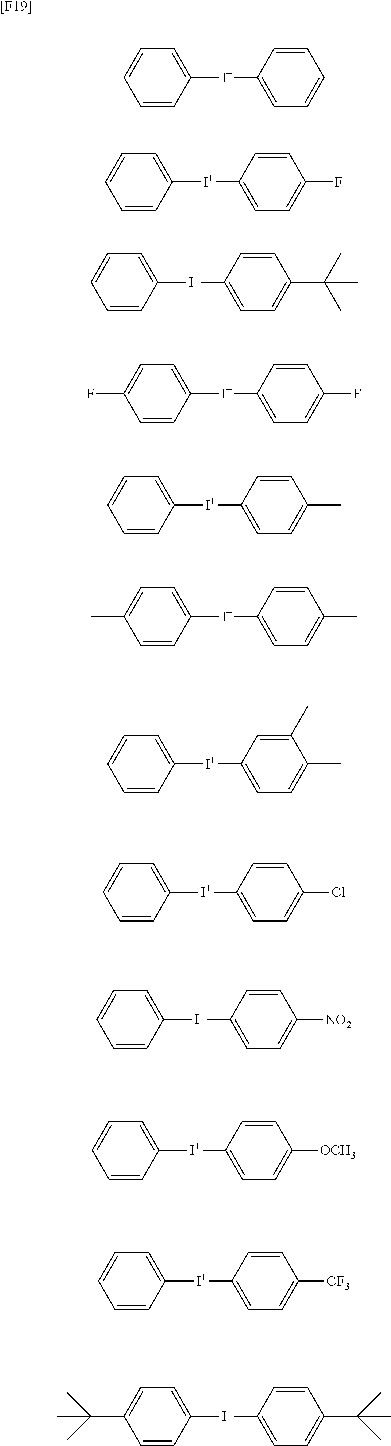

In polymer [A], the relative amount of structural unit (I) in all the structural units forming polymer [A] is preferably 2 mol % or more, more preferably 5 mol % or more, still more preferably 10 mol % or more. Also, the relative amount of structural unit (I) in all the structural units forming polymer [A] is preferably 50 mol % or less, more preferably 40 mol % or less, still more preferably 30 mol % or less. Adjusting the structural unit (I) content to satisfy the aforementioned conditions is preferred, since the sensitivity of the present composition can be further enhanced, and excessive dissolution of the light-exposed part to a developer can be suppressed, to thereby further expand the process window.

### [Additional Structural Unit]

Polymer [A] may further contain a structural unit other than the structural unit (I) (hereinafter may also be referred to as an “additional structural unit”). Examples of the additional structural unit include the following structural units (II) to (V).

### Structural Unit (II)

Polymer [A] may contain a structural unit (II) having an acid-releasable group. The acid-releasable group refers to a group which can substitute a hydrogen atom of an acidic group such as a carboxy group or a hydroxy group and which is eliminated by the action of acid. When polymer [A] has an acid-releasable group, the acid-releasable group is released from the present composition via exposure to light to thereby form an acidic group, which modifies the solubility of the polymer component(s) in a developer. As a result, excellent lithographic characteristics can be imparted to the present composition.

No particular limitation is imposed on the structural unit (II), so long as the unit has an acid-releasable group. Examples of the structural unit (II) include structural units represented by formula (ii-1) (hereinafter may also be referred to as “structural units (II-1)”), structural units represented by formula (ii-2) (hereinafter may also be referred to as “structural units (II-2)”), and structural units represented by formula (ii-3) (hereinafter may also be referred to as “structural units (11-3)”).

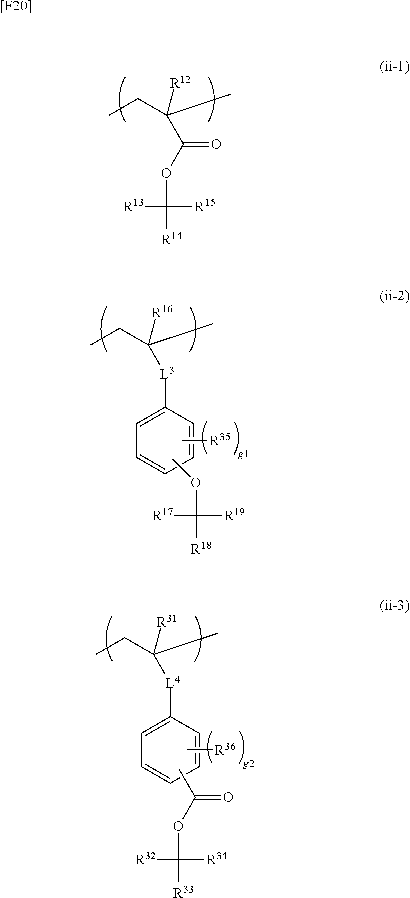

(In formula (ii-1), R¹² represents a hydrogen atom, a fluorine atom, a methyl group, or a trifluoromethyl group; R¹³ represents a C1 to C20 monovalent hydrocarbon group; each of R¹⁴ and R¹⁵ represents a C1 to C20 monovalent hydrocarbon group, or a C3 to C20 aliphatic ring structure formed with a carbon atom to which a linked moiety of two of R¹⁴ and R¹⁵ is bonded;
in formula (ii-2), R¹⁶ represents a hydrogen atom or a methyl group; L³ represents a single bond, —COO—, or —CONH—; each of R¹⁷, R¹⁸, and R¹⁹ represents a hydrogen atom, a C1 to C20 monovalent hydrocarbon group, or a C1 to C20 monovalent oxyhydrocarbon group; R³⁵ represents a monovalent substituent; and g1 is an integer of 0 to 4; and
in formula (ii-3), R³¹ represents a hydrogen atom or a methyl group; L⁴ represents a single bond, —COO—, or —CONH—; each of R³², R³³, and R³⁴ represents a hydrogen atom, a C1 to C20 monovalent hydrocarbon group, or a C1 to C20 monovalent oxyhydrocarbon group; R³⁶ represents a monovalent substituent; and g2 is an integer of 0 to 4.)

In formula (ii-1), from the viewpoint of copolymerization performance of the monomer for providing the structural unit (II-1), R¹² is preferably a hydrogen atom or a methyl group, with a methyl group being more preferred. In formula (ii-2), from the viewpoint of copolymerization performance of the monomer for providing the structural unit (II-2), R¹⁶ is preferably a hydrogen atom. Similarly, R³¹ in formula (ii-3) is preferably a hydrogen atom.

Examples of the C1 to C20 monovalent hydrocarbon group represented by each of R¹³ to R¹⁵, R¹⁷ to R¹⁹, and R³² to R³⁴ include a C1 to C20 monovalent chain hydrocarbon group, a C3 to C20 monovalent alicyclic hydrocarbon group, and a C6 to C20 monovalent aromatic hydrocarbon group. Specific examples thereof include the same groups as exemplified in relation to the monovalent hydrocarbon group represented by each of R⁵⁰, R⁵¹, and R⁵² in formula (Y-1) to formula (Y-3).

Examples of the C3 to C20 aliphatic ring structure formed with a carbon atom to which a linked moiety of two of R¹⁴ and R¹⁵ is bonded include monocyclic aliphatic ring structures such as a cyclopropane structure, a cyclobutane structure, a cyclopentane structure, a cyclohexane structure, a cycloheptane structure, a cyclooctane structure; and polycyclic aliphatic ring structures such as a norbornane structure, an adamantane structure, a tricyclodecane structure, and a tetracyclododecane structure.

Examples of the C1 to C20 monovalent oxyhydrocarbon group represented by each of R¹⁷ to R¹⁹, and R³² to R³⁴ include similar groups as exemplified in relation to the C1 to C20 monovalent hydrocarbon group represented by any of the aforementioned R¹³ to R¹⁵, R¹⁷ to R¹⁹, and R³² to R³⁴ which groups have an oxygen atom at the terminal on the chemical bond side. Of these, the C1 to C20 monovalent oxyhydrocarbon group represented by each of R¹⁷ to R¹⁹, and R³² to R³⁴ is preferably an alkoxy group, a cycloalkoxy group, or a cycloalkylalkoxy group.

Examples of the monovalent substituent represented by each of R³⁵ and R³⁶ include a C1 to C3 alkyl group, a C1 to C3 alkoxy group, a hydroxy group, and a halogen atom. The numbers “g1” and “g2” are preferably 0 to 2, more preferably 0 or 1.

Specific examples of the structural unit (II-1) include structural units represented by the following formulas.

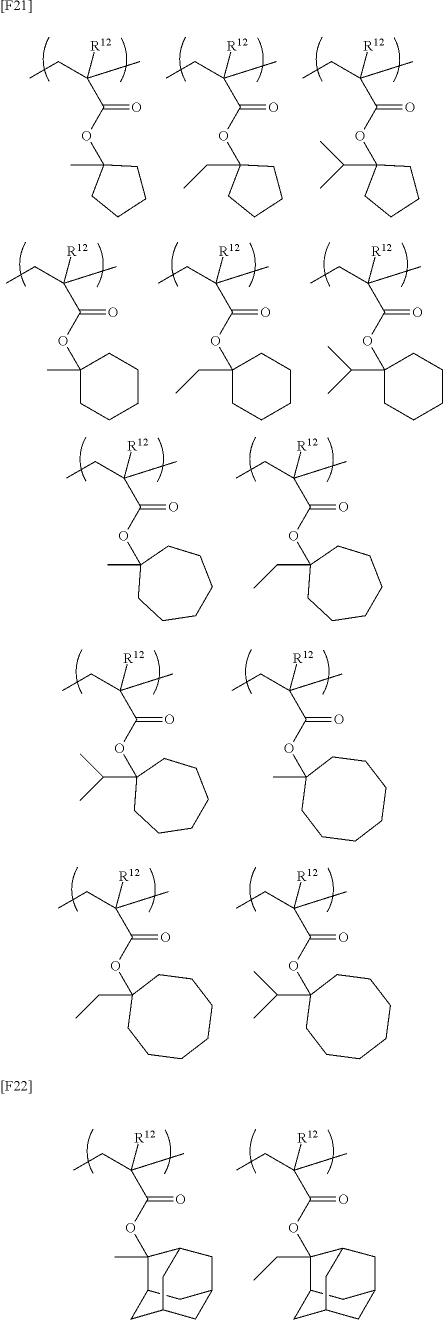

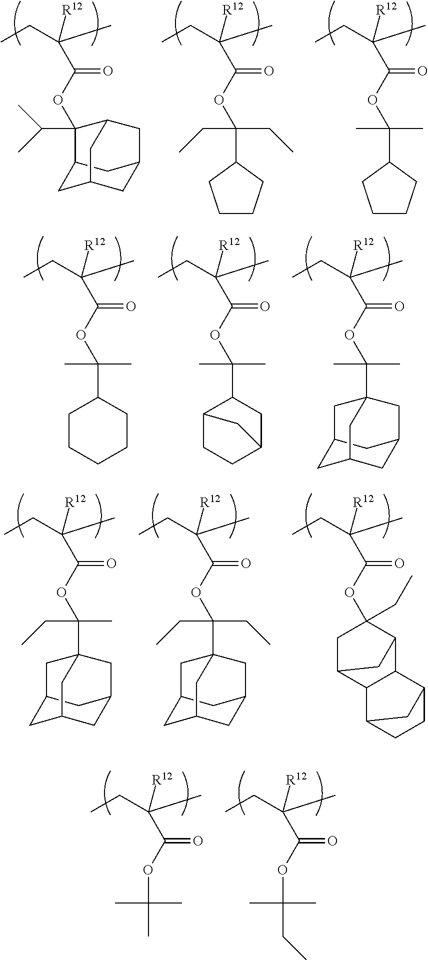

(In the above formulas, R¹² represents a hydrogen atom, a fluorine atom, a methyl group, or a trifluoromethyl group.)

Specific examples of the structural unit (II-2) include structural units represented by the following formulas.

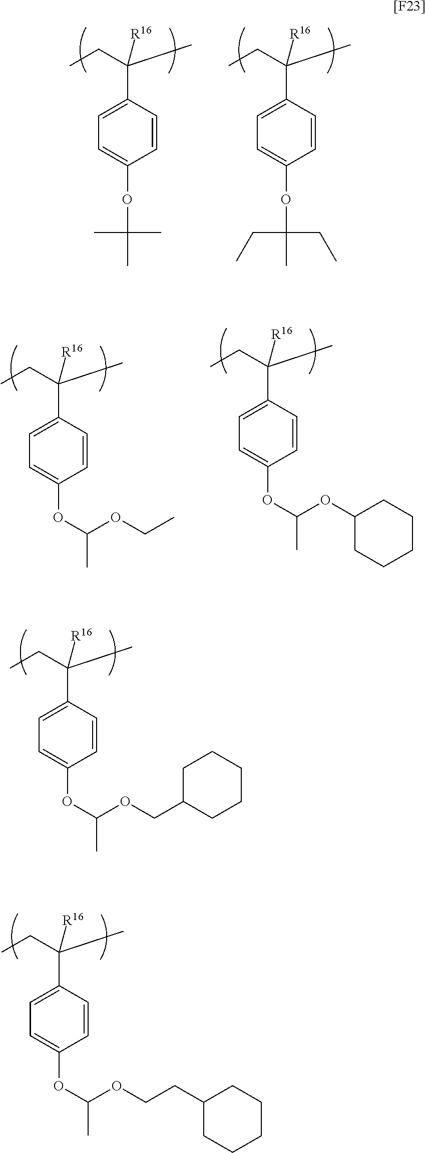

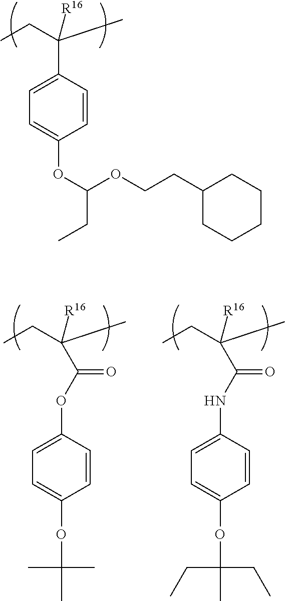

(In the above formulas, R¹⁶ represents a hydrogen atom or a methyl group.)

Specific examples of the structural unit (II-3) include structural units represented by the following formulas.

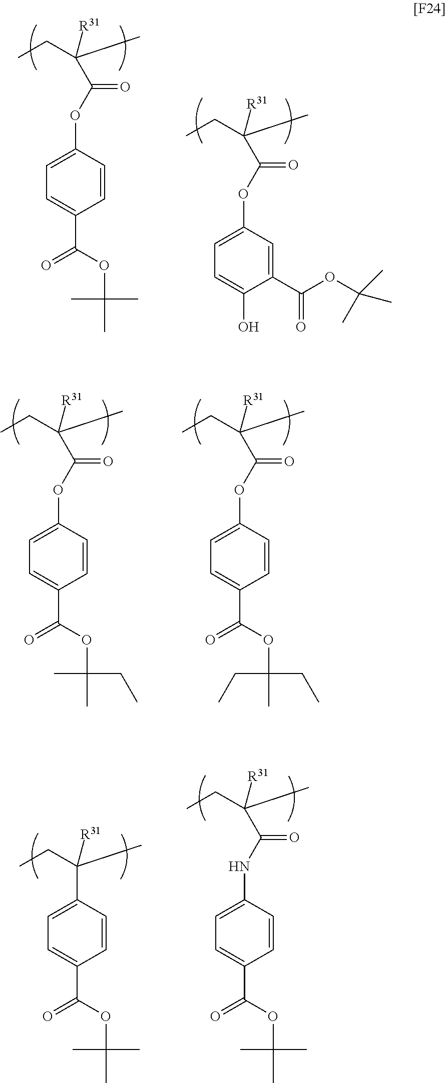

(In the above formulas, R³¹ represents a hydrogen atom or a methyl group.)

When polymer [A] contains the structural unit (II), the relative amount of structural unit (II) in all the structural units forming polymer [A] is preferably 20 mol % or more, more preferably 25 mol % or more, still more preferably 30 mol % or more. Also, the relative amount of structural unit (II) in all the structural units forming polymer [A] is preferably 80 mol % or less, more preferably 75 mol % or less, still more preferably 70 mol % or less. Adjusting the structural unit (II) content to satisfy the aforementioned conditions is preferred, since considerable difference in dissolution rate with respect to a developer between the exposed part and the unexposed part can be sufficiently attained, whereby favorable resist film pattern can be provided.

### Structural Unit (III)

Preferably, polymer [A] further contains a structural unit having a hydroxy group which is bonded to an aromatic ring (hereinafter may also be referred to as “structural unit (III)”). Incorporation of the structural unit (III) into polymer [A] is preferred, since lithographic characteristics of the present composition (e.g., line width roughness (LWR) performance and critical dimension uniformity (CDU) performance) can be enhanced, and dissolution of the light-unexposed part to a developer can be suppressed, to thereby sufficiently reduce development failure.

In the structural unit (III), examples of the aromatic ring to which a hydroxy group is bonded include a benzene ring, a naphthalene ring, and an anthracene ring. Of these, a benzene ring or a naphthalene ring is preferred, with a benzene ring being more preferred. No particular limitation is imposed on the number of the hydroxy group(s) bonded to the aromatic ring, and the number is preferably 1 to 3, more preferably 1 or 2. Examples of the structural unit (III) include structural units represented by the following formula (iii).

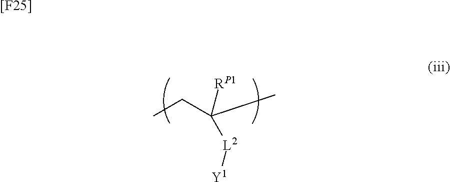

(In formula (iii), R^{P1} represents a hydrogen atom, a fluorine atom, a methyl group, or a trifluoromethyl group; L² represents a single bond, —O—, —CO—, —COO—, or —CONH—; and Y¹ represents a monovalent group having a hydroxy group which is bonded to an aromatic ring.)

From the viewpoint of copolymerization performance, the monomer forming the structural unit (III), R^{P1} in formula (iii) is preferably a hydrogen atom or a methyl group. L² is preferably a single bond or —COO—. In the case where a polymer containing the structural unit (III) is produced as polymer [A], the polymer containing the structural unit (III) may be formed by conducting polymerization while a phenolic hydroxy group is protected by a protective group (e.g., an alkali-releasing group) and then performing hydrolysis for deprotection.

Specific examples of the structural unit (III) include structural units represented by the following formulas.

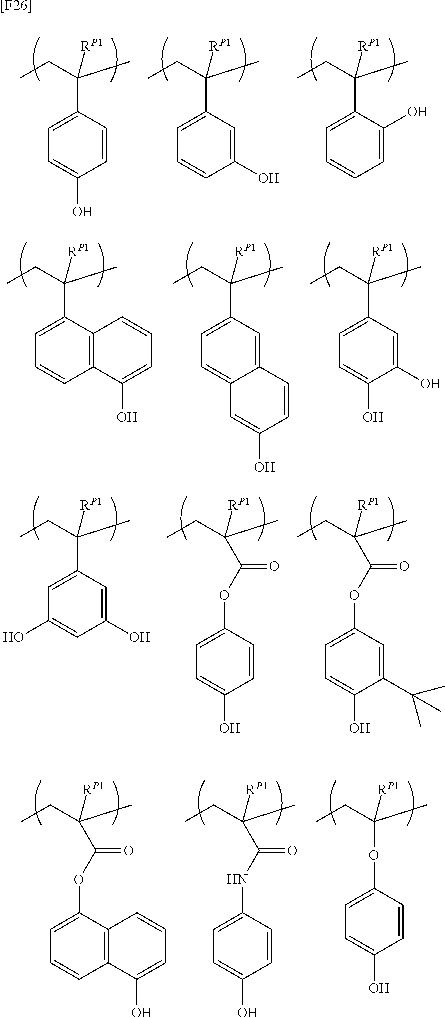

(In the above formulas, R^{P1} represents a hydrogen atom, a fluorine atom, a methyl group, or a trifluoromethyl group.)

When polymer [A] contains the structural unit (III), the relative amount of structural unit (III) in all the structural units forming polymer [A] is preferably 5 mol % or more, more preferably 10 mol % or more, still more preferably 15 mol % or more. Also, the relative amount of structural unit (III) in all the structural units forming polymer [A] is preferably 90 mol % or less, more preferably 80 mol % or less, still more preferably 60 mol % or less. Adjusting the structural unit (III) content to satisfy the aforementioned conditions is preferred, since the lithographic characteristics of the present composition can be further enhanced.

Notably, polymer [A] may contain a structural unit having both an acid-releasable group and a hydroxy group which is bonded to an aromatic ring. In the present specification, the structural unit having both an acid-releasable group and a hydroxy group which is bonded to an aromatic ring is categorized into structural unit (II).

### Structural Unit (IV)

Polymer [A] may further contain a structural unit having a lactone structure, a cyclic carbonate structure, a sultone structure, or a combination of two or more members thereof (hereinafter may also be referred to as “structural unit (IV)”). Incorporation of the structural unit (IV) into polymer [A] is preferred, since solubility of the composition in a developer can be controlled, whereby lithographic characteristics of the present composition can be further enhanced. Also, the presence of the structural unit (IV) in polymer [A] can improve close adhesion between a substrate and a resist film formed from the present composition.

Specific examples of the structural unit (IV) include structural units represented by the following formulas.

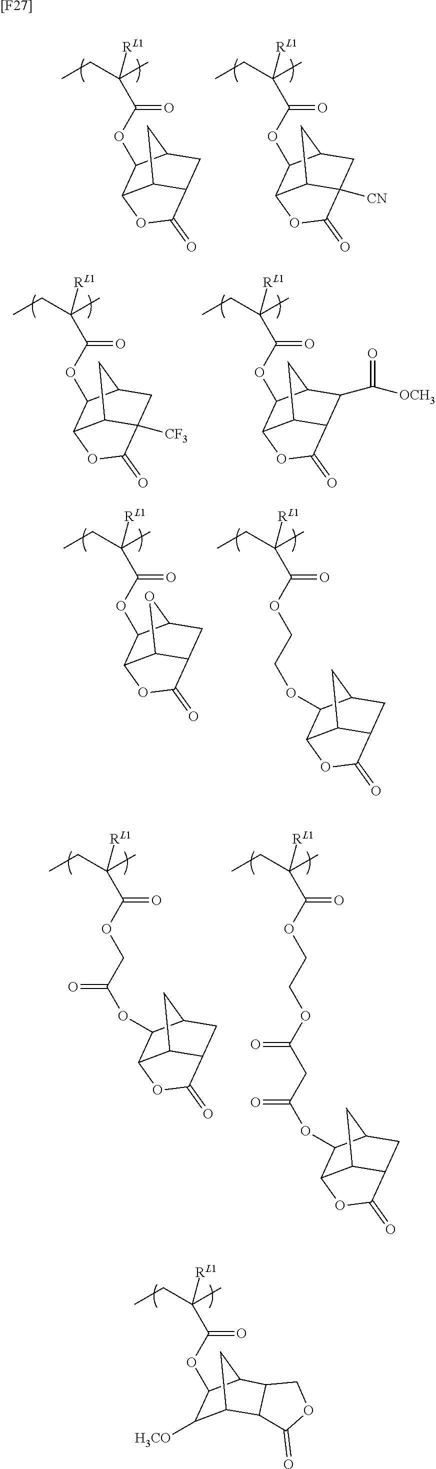

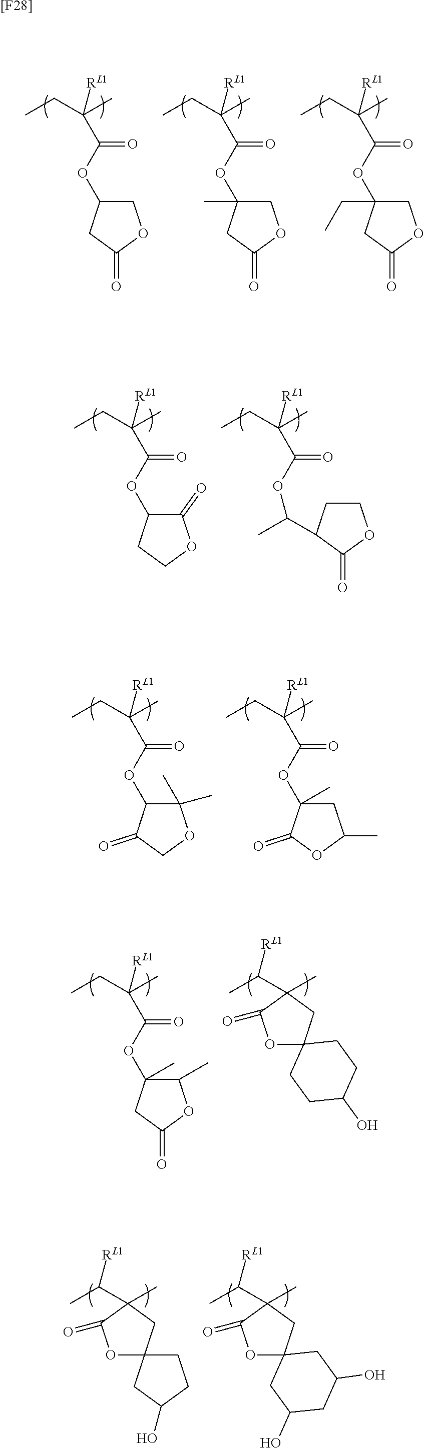

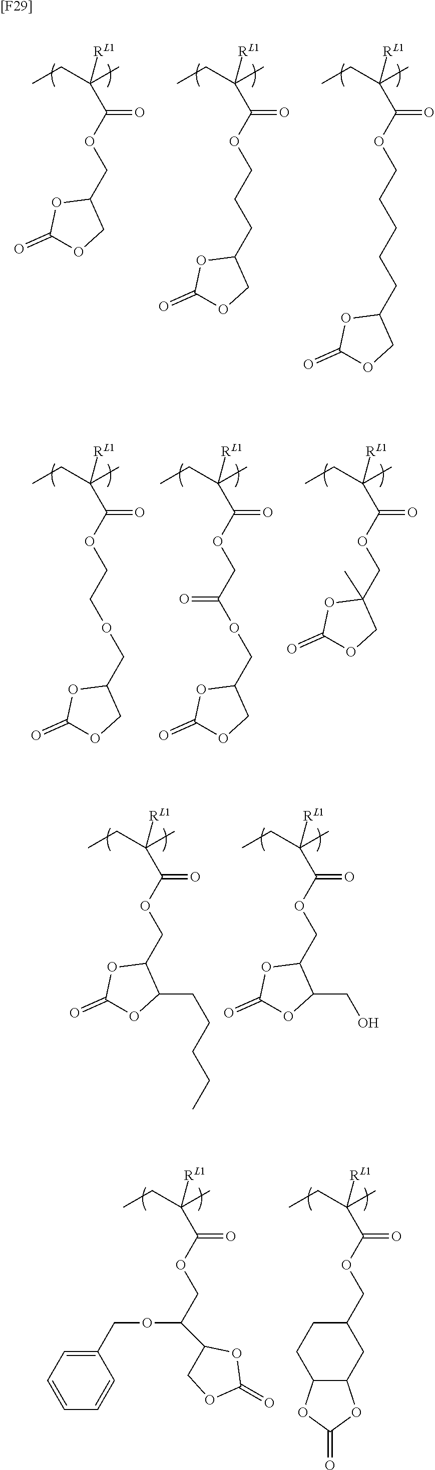

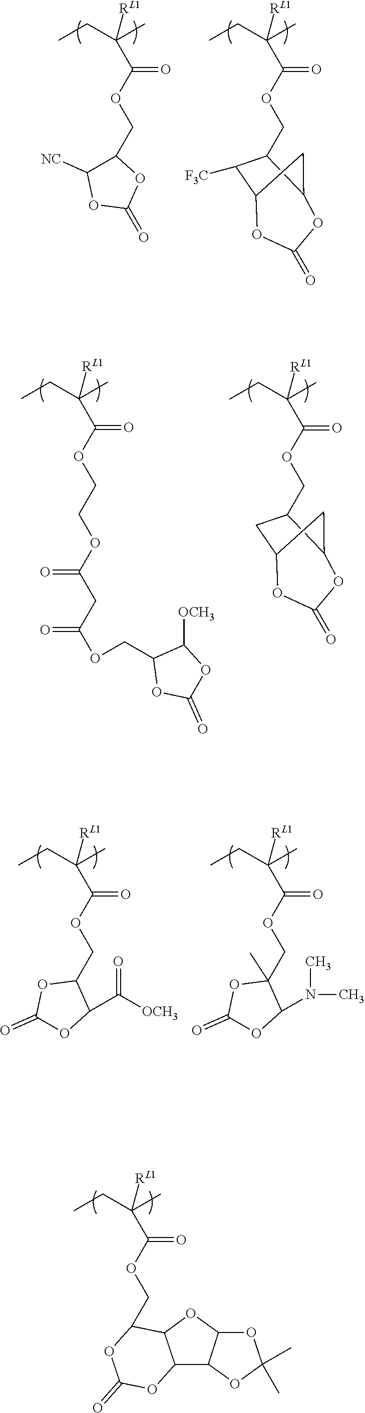

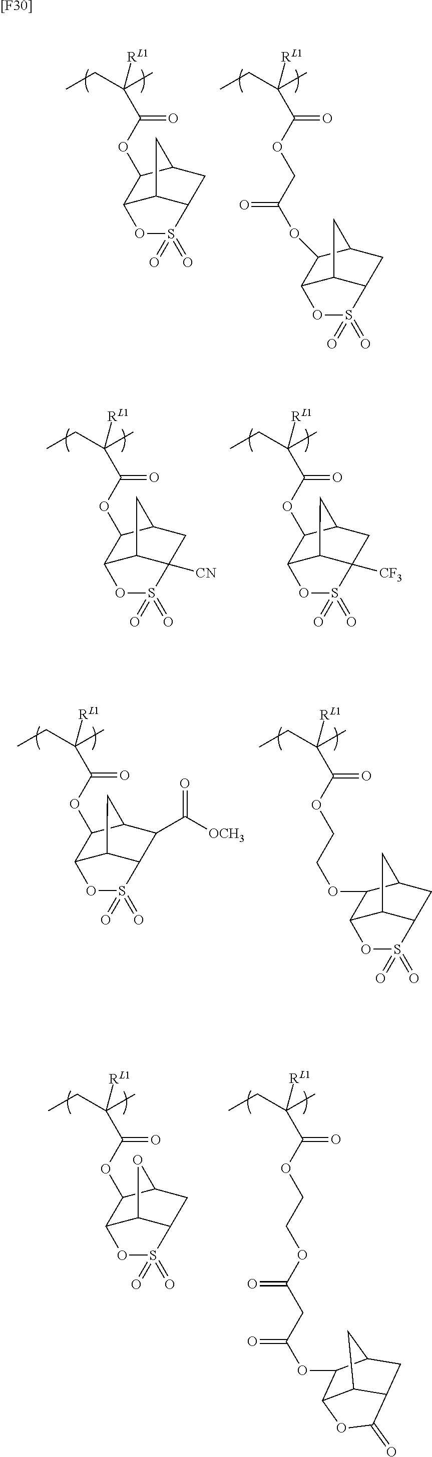

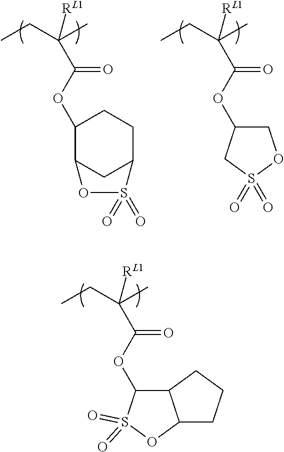

(In the above formulas, R^{L1} represents a hydrogen atom, a fluorine atom, a methyl group, or a trifluoromethyl group.)

When polymer [A] contains the structural unit (IV), the relative amount of structural unit (IV) in all the structural units forming polymer [A] is preferably 1 mol % or more, more preferably 3 mol % or more, still more preferably 5 mol % or more. Also, the relative amount of structural unit (IV) in all the structural units forming polymer [A] is preferably 50 mol % or less, more preferably 30 mol % or less, still more preferably 15 mol % or less. Adjusting the structural unit (IV) content to satisfy the aforementioned conditions is preferred, since lithographic characteristics of the present composition as well as the close adhesion between a substrate and a resin film formed from the present composition can be enhanced.

### Structural Unit (V)

Polymer [A] may further contain a structural unit having an alcoholic hydroxy group (except for the cases corresponding to structural units (I) to (IV)) (hereinafter may also be referred to as a “structural unit (V)”). As used herein, the term “alcoholic hydroxy group” refers to a group having a structure in which a hydroxy group is directly bonded to an aliphatic hydrocarbon group. The aliphatic hydrocarbon group may be a chain hydrocarbon group or an alicyclic hydrocarbon group. Incorporation of the structural unit (V) into polymer [A] is preferred, since the solubility of the component(s) in a developer can be improved, whereby lithographic characteristics can be further improved.

The structural unit (V) is preferably derived from an unsaturated monomer having an alcoholic hydroxy group. Examples of the structural unit (V) include structural units represented by the following formulas.

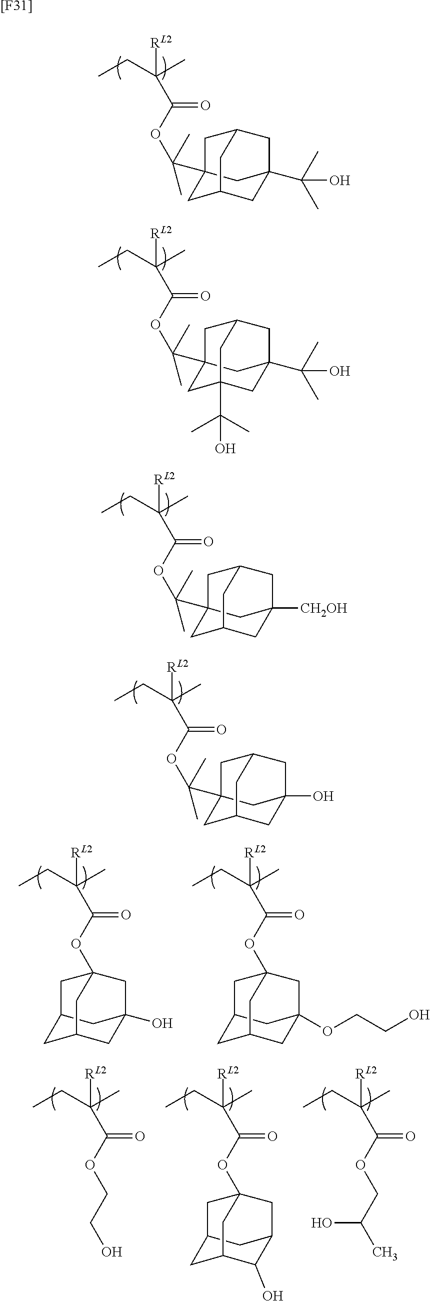

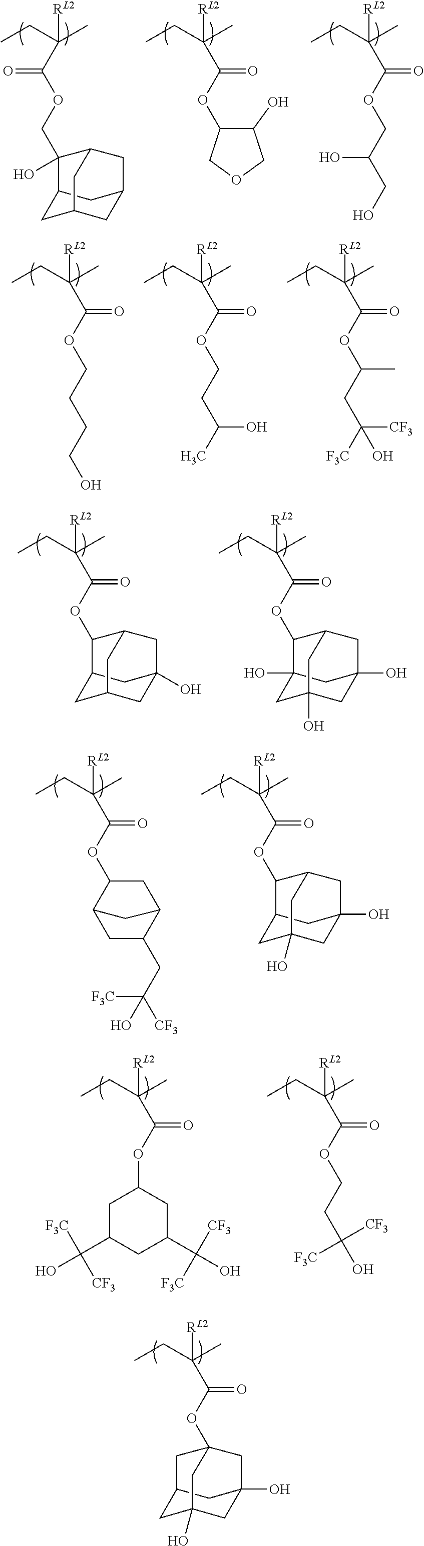

(In the above formulas, R^{L2} represents a hydrogen atom, a fluorine atom, a methyl group, or a trifluoromethyl group.)

When polymer [A] contains structural unit (V), the relative amount of structural unit (V) in all the structural units forming polymer [A] is preferably 1 mol % or more, more preferably 3 mol % or more. Also, the relative amount of structural unit (V) in all the structural units forming polymer [A] is preferably 30 mol % or less, more preferably 20 mol % or less.

In addition to the aforementioned additional structural units, the following structural units (VI) and (VII) may be acceptable.

Structural Unit (VI) Having a Cyano Group, a Nitro Group, or a Sulfonamide Group (e.g., a Structural Unit Derived from 2-cyanomethyladamantane-2-yl (meth)acrylate)
Structural Unit (VII) Having a Non-Acid-Releasable Hydrocarbon Group (e.g., a Structural Unit Derived from Styrene, a Structural Unit Derived from Vinylnaphthalene, or a Structural Unit Derived from n-pentyl (meth)acrylate)

The relative amounts of these structural units may be appropriately set individually, so long as the effect of the present disclosure are not impaired.

In the present composition, the polymer [A] content with respect to the entire solid content of the present composition is preferably 50 mass % or more, more preferably 55 mass % or more, still more preferably 60 mass % or more. Also, the polymer [A] content with respect to the entire solid content of the present composition is preferably 99 mass % or less, more preferably 98 mass % or less, still more preferably 95 mass % or less. Generally, polymer [A] serves as a base resin of the present composition. As used herein, the term “base resin” refers to a polymer component having a content of 50 mass % or more with respect to the entire solid content of the present composition. The present composition may contain one species or two or more species of polymer [A]. The “solid content” refers to the components contained in the present composition other than solvent [D].

### <Synthesis of Polymers>

Polymer [A] may be synthesized through, for example, polymerizing monomers which provide corresponding structural units in the presence of a radical polymerization initiator or the like in an appropriate solvent.

Examples of the radical polymerization initiator include azo-type radical initiators such as azobisisobutyronitrile (AIBN), 2,2′-azobis(4-methoxy-2,4-dimethylvaleronitrile), 2,2′-azobis(2-cyclopropylpropionitrile), 2,2′-azobis(2,4-dimethylvaleronitrile), dimethyl 2,2′-azobisisobutyrate, and 2,2′-azobis(methyl isoburylate); and peroxide-type radical initiators such as benzoyl peroxide, t-butyl hydroperoxide, and cumene hydroperoxide. Of these, azo-type radical initiators are preferred. These radical polymerization initiators may be used singly or in combination of two or more species.

Examples of the solvent employed in polymerization include alkanes such as n-pentane, n-hexane, n-heptane, n-octane, n-nonane, and n-decane; cycloalkalnes such as cyclohexane, cycloheptane, cyclooctane, decalin, and norbornane; aromatic hydrocarbons such as benzene, toluene, xylene, ethylbenzene, and cumene; halo-hydrocarbons such as chlorobutanes, bromohexanes, dichloroethanes, hexamethylene dibromide, and chlorobenzene; saturated carboxylic acid esters such as ethyl acetate, n-butyl acetate, i-butyl acetate, and methyl propionate; ketones such as acetone, butanone, 4-methyl-2-pentanone, and 2-heptanone; ethers such as tetrahydrofuran, dimethoxyethanes, and diethoxyethanes; and alcohols such as methanol, ethanol, 1-propanol, 2-propanol, and 4-methyl-2-pentanol. These solvents employed in polymerization may be used singly or in combination of two or more species.

The reaction temperature in polymerization is preferably 40° C. or higher, more preferably 50° C. or higher. Also, the reaction temperature is preferably 150° C. or lower, more preferably 120° C. or lower. The reaction time in polymerization is preferably 1 hour or longer, more preferably 2 hours or longer. Also, the reaction time is preferably 48 hours or shorter, more preferably 24 hours or shorter.

The weight average molecular weight (Mw) of polymer [A], which is determined through gel permeation chromatography (GPC) and is reduced to polystyrene, is preferably 1,000 or more, more preferably 2,000 or more, still more preferably 3,000 or more, yet more preferably 4,000 or more. Also, the Mw of polymer [A] is preferably 50,000 or less, more preferably 30,000 or less, still more preferably 20,000 or less, yet more preferably 15,000 or less. Adjusting the Mw of polymer [A] so as to satisfy the above conditions is preferred, since coatability of the present composition can be improved, and development failure can be sufficiently suppressed.

The ratio (Mw/Mn) of Mw to the number average molecular weight (Mn) of polymer [A], which is determined through gel permeation chromatography (GPC) and is reduced to polystyrene, is preferably 5.0 or less, more preferably 3.0 or less, still more preferably 2.0 or less, yet more preferably 1.8 or less. Also, the Mw/Mn of polymer [A] is generally 1 or more, preferably 1.3 or more.

<Synthesis of Compounds (a1) and (a2)>

Both compounds (a1) and (a2) may be synthesized through customary methods of organic chemistry in appropriate combinations. In one procedure, an aldehyde compound having a (meth)acryloyl group or a vinylphenyl group depending on R² in formulas (4) and (5) is reacted with a diol compound having a partial structure corresponding to R⁴ in formulas (4) and (5) under acidic conditions. In another procedure, a diol compound having a (meth)acryloyl group or a vinylphenyl group is reacted with an aldehyde compound having a partial structure corresponding to R⁴ in formulas (4) and (5) under acidic conditions. Needless to say, the method of synthesizing compound (a1) or (a2) is not limited to the above procedures.

### <[B] Acid Generator>

Acid generator [B] is a substance which can generate acid by exposing the present composition to light. A typical example of acid generator [B] is an onium salt composed of an onium cation and an organic anion. In an alternative mode, polymer [A] and acid generator [B] are added to the present composition, and an acid-releasable group among the polymer components is released by polymer [A] and an acid generated by acid generator [B] (preferably, a strong acid such as sulfonic acid, imidic acid, or a methide acid), to thereby provide an acidic group, which can modify the solubility of the polymer component(s) in a developer.

No particular limitation is imposed on acid generator [B] to be incorporated into the present composition, and any known acid generator for forming a resist pattern may be employed. The onium cation included in acid generator [B] is preferably a radiation-sensitive onium cation. Among them, from the viewpoint of enhancing the lithographic characteristics of the present composition, the onium cation is preferably a sulfonium cation or an iodonium cation. Examples thereof include cations represented by formulas (7), (8), and (9).

No particular limitation is imposed on the organic anion included in acid generator [B]. Examples of the anion include organic anions each having a sulfonate anion structure, an imido anion structure, or a methido anion structure. Of these, an organic anion having a sulfonate anion structure is preferred. More specifically, the organic anions represented by formula (11) are preferably used.

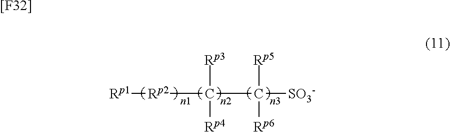

(In formula (11), R^{p1} represents a monovalent group having a ≥5-membered ring structure; R^{p2} represents a divalent linkage group; each of R^{p3} and R^{p4} represents a hydrogen atom, a fluoro group, a C1 to C20 monovalent hydrocarbon group, or a C1 to C20 monovalent fluorinated hydrocarbon group; each of R^{p5} and R^{p6} represents a hydrogen atom, a fluoro group, or a C1 to C20 monovalent fluorinated hydrocarbon group; n1 is an integer of 0 to 10; n2 is an integer of 0 to 10; n3 is an integer of 1 to 10; n1+n2+n3 is 1 to 30; when n1 is 2 or greater, a plurality of R^{p2} are identical to or different from one another; when n2 is 2 or greater, a plurality of R^{p3} are identical to or different from one another, and a plurality of R^{p4} are identical to or different from one another; when n3 is 2 or greater, a plurality of R^{p5} are identical to or different from one another, and a plurality of R^{p6} are identical to or different from one another; when n3 is 1, neither R^{p5} or R^{p6} is a hydrogen atom; and when n3 is 2 or greater, neither R^{p5} or R^{p6} is a hydrogen atom.)

In formula (11), examples of the monovalent group having a ≥5-membered ring structure represented by R^{p1} include a monovalent group having ≥5-membered alicyclic hydrocarbon structure, a monovalent group having ≥5-membered aliphatic heterocyclic structure, a monovalent group having ≥5-membered aromatic ring structure, and a monovalent group having ≥5-membered aromatic heterocyclic structure.

Examples of the ≥5-membered alicyclic hydrocarbon structure include monocyclic cycloalkane structures such as a cyclopentane structure, a cyclohexane structure, a cycloheptane structure, a cyclooctane structure, a cyclononane structure, a cyclodecane structure, and a cyclododecane structure; monocyclic cycloalkene structures such as a cyclopentene structure, a cyclohexene structure, a cycloheptene structure, a cyclooctene structure, and a cyclodecene structure; polycyclic cycloalkane structures such as a norbornane structure, an adamantane structure, a tricyclodecane structure, and a tetracyclododecane structure; and polycyclic cycloalkene structures such as a norbornene structure and a tricyclodecene structure.

Examples of the ≥5-membered aliphatic heterocyclic structure include lactone structures such as a hexanolactone structure and a norbornane lactone structure; sultone structures such as a hexanosultone structure and a norbornane sultone structure; oxygen-atom-containing heterocyclic structures such as an oxacycloheptane structure, an oxanorbornane structure, and a cyclic acetal structure; nitrogen-atom-containing heterocyclic structures such as an azacyclohexane structure and a diazabicyclooctane structure; and sulfur-atom-containing heterocyclic structures such as a thiacyclohexane structure and a thianorbornane structure.

Examples of the ≥5-membered aromatic ring structure include a benzene structure, a naphthalene structure, a phenanthrene structure, and an anthracene structure.

Examples of the ≥5-membered aromatic heterocyclic structure include oxygen-atom-containing heterocyclic structures such as a furan structure, a pyran structure, a banzopyran structure; and nitrogen-atom-containing heterocyclic structures such as a pyridine structure, a pyrimidine structure, and an indole structure.

Notably, the hydrogen atoms present in the ring structure of R^{p1} may be partially or totally substituted by a substituent. Examples of the substituent include a fluoro group, a chloro group, a bromo group, an iodo group, a hydroxy group, a carboxy group, a cyano group, a nitro group, an alkoxy group, an alkoxycarbonyl group, an alkoxycarbonyloxy group, an acyl group, and an acyloxy group. Of these, R^{p1} is preferably a ≥6-membered aromatic ring structure in which at least a part of hydrogen atoms are substituted by an iodo group.

Examples of the divalent linkage group represented by R^{p2} include a carbonyl group, an ether group, a carbonyloxy group, a sulfide group, a thiocarbonyl group, a sulfonyl group, and a divalent hydrocarbon group. Of these, a carbonyloxy group, a sulfonyl group, an alkanediyl group, and a cycloalkanediyl group are preferred, with a carbonyloxy group, a sulfonyl group, and a cycloalkanediyl group being more preferred. Further, a carbonyloxy group, a sulfonyl group, and a norbornanediyl group are still more preferred.

Examples of the C1 to C20 monovalent hydrocarbon group represented by R^{p3} or R^{p4} include a C1 to C20 alkyl group. Examples of the C1 to C20 monovalent fluorinated hydrocarbon group represented by R^{p3} or R^{p4} include a C1 to C20 fluorinated alkyl group. As R^{p3} and R^{p4}, a hydrogen atom, a fluoro group, and a fluoroalkyl group are preferred, with a fluoro group and a perfluoroalkyl group being more preferred. Further a fluoro group and a trifluoromethyl group are still more preferred.

Examples of the C1 to C20 monovalent fluorinated hydrocarbon group represented by R^{p5} or R^{p6} include a C1 to C20 fluoroalkyl group. As R^{p5} and R^{p6}, a fluoro group and a fluoroalkyl group are preferred, with a fluoro group and a perfluoroalkyl group being more preferred. Further, a fluoro group and a trifluoromethyl group are still more preferred, with a fluoro group being particularly preferred. When n3 is 1, each of R^{p5} and R^{p6} is preferably a fluoro group, or, also preferably, R^{p5} is a fluoro group and R^{p6} is a trifluoromethyl group.

The number “n1” is preferably 0 to 5, more preferably 0 to 3, still more preferably 0 to 2, particularly preferably 0 or 1. The number “n2” is preferably 0 to 5, more preferably 0 to 2, still more preferably 0 or 1, particularly preferably 0. The number “n3” is preferably 1 to 5, more preferably 1 to 3, still more preferably 1 or 2. Adjusting the n3 to satisfy the above conditions is preferred, since the strength of the acid generated by acid generator [B] can be enhanced, whereby defect suppression performance, LWR performance, and sensitivity of the present composition can be further enhanced. The value “n1+n2+n3” is preferably 2 or more, and preferably 10 or less, more preferably 5 or less.

Specific examples of acid generator [B] include compounds represented by the following formulas. However, acid generator [B] is not limited to the following structures.

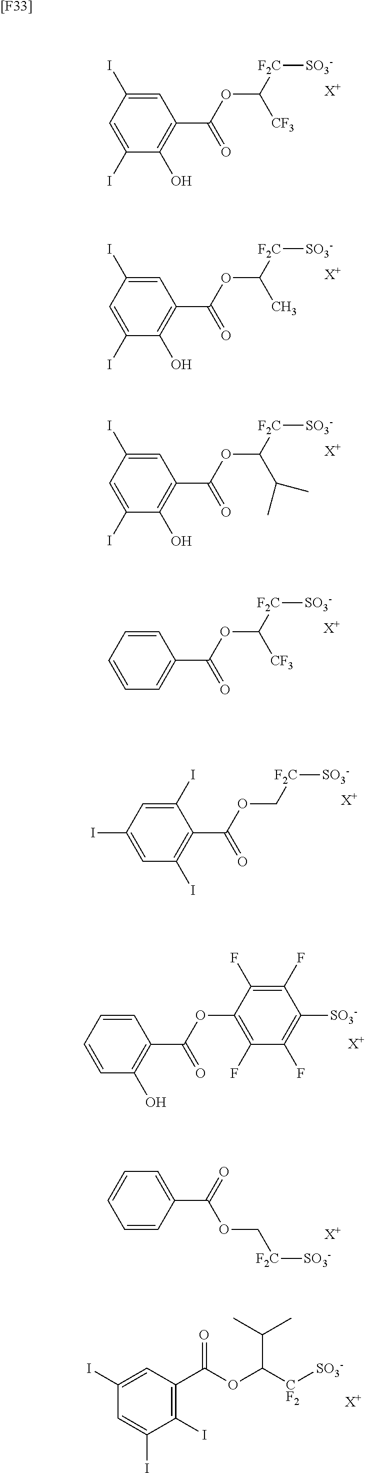

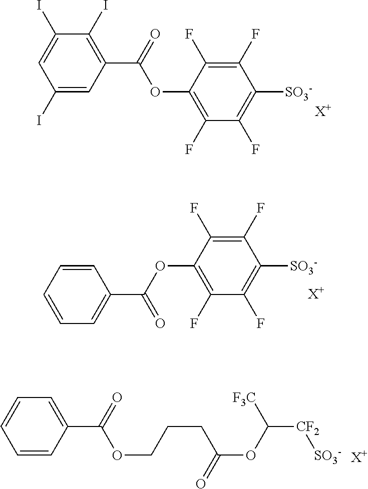

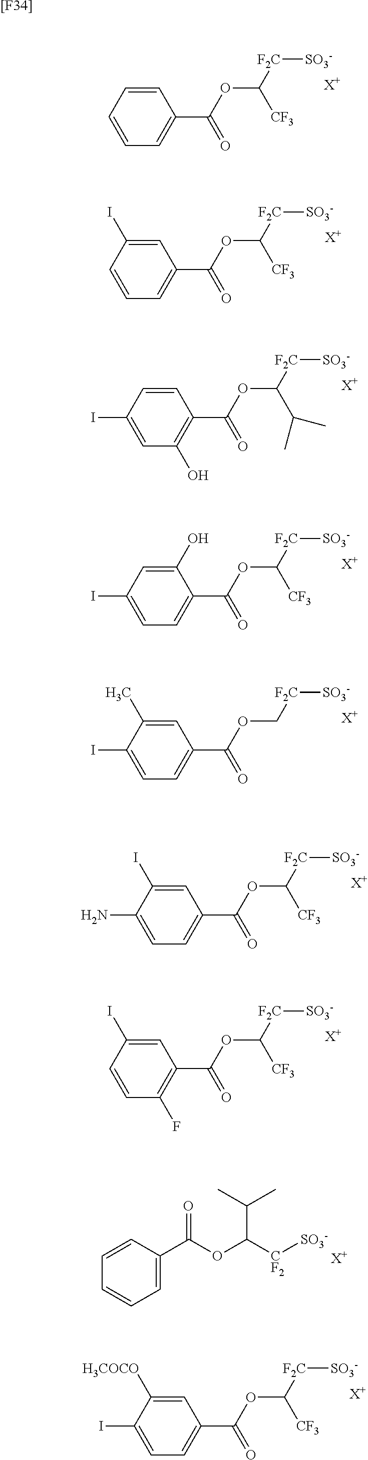

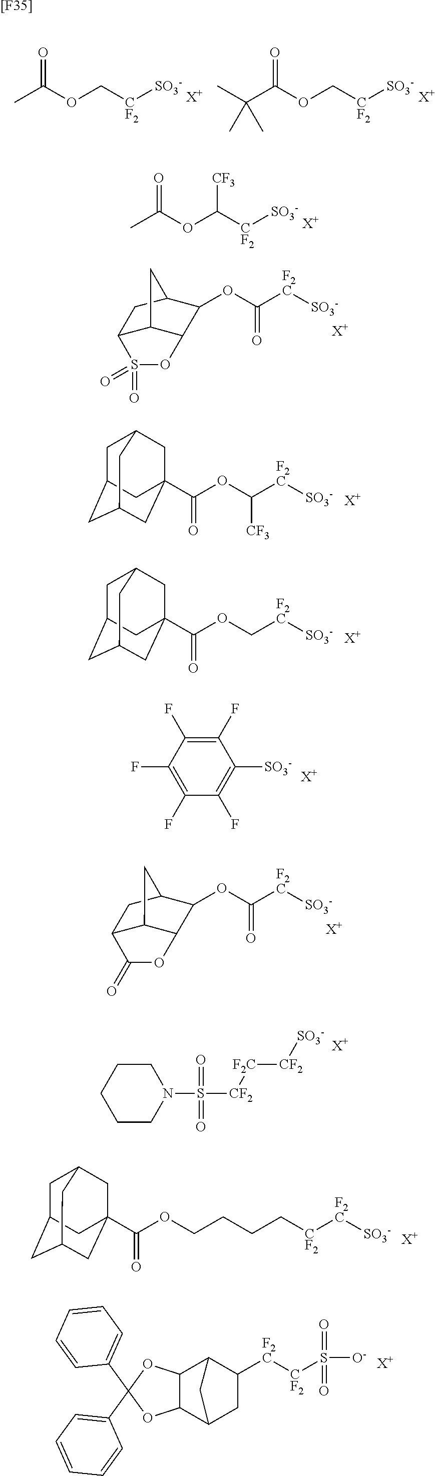

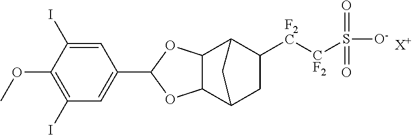

(In the above formulas, X⁺ represents a cation represented by formula (7), (8), or (9)).

In the present composition, the relative amount of acid generator [B] with respect to 100 parts by mass of polymer [A] is preferably 20 mass % or less, more preferably 15 mass % or less. Adjusting the acid generator [B] content to satisfy the above conditions is preferred, since the sensitivity of the present composition can be enhanced, and excessive dissolution of the light-exposed part to a developer can be suppressed, to thereby further expand the process window. Acid generator [B] may be used singly or in combination of two or more species.

### <[C] Acid Diffusion Control Agent>

Acid diffusion control agent [C] is incorporated into the present composition in order to suppress diffusion of an acid generated by light exposure to the composition in the resist film, to thereby suppress chemical reaction, which would otherwise be caused by the acid, in a non-exposed area. Incorporation of acid diffusion control agent [C] into the present composition is preferred, from the viewpoint of enhancement in lithographic characteristics of the present composition. Furthermore, variation in line width of a resist pattern, which would otherwise be caused by variation in post-exposure time (i.e., duration of time from light exposure to development) can be suppressed, whereby a radiation-sensitive composition having excellent process stability can be obtained. Examples of acid diffusion control agent [C] include a nitrogen-containing compound and a light-degradable base.

### Nitrogen-Containing Compound

Examples of the nitrogen-containing compound include compounds represented by formula (12) (hereinafter may also be referred to as “nitrogen-containing compounds (12A)”), a compound having two nitrogen atoms (hereinafter may also be referred to as a “nitrogen-containing compound (12B)”), a compound having three nitrogen atoms (hereinafter may also be referred to as a “nitrogen-containing compound (12C)”), an amido group-containing compound, a urea compound, a nitrogen-containing heterocyclic compound, and a nitrogen-containing compound having an acid-releasable group.

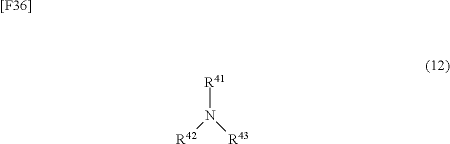

(In formula (12), each of R⁴¹, R⁴², and R⁴³ represents a hydrogen atom, a substituted or non-substituted alkyl group, a substituted or non-substituted cycloalkyl group, a substituted or non-substituted aryl group, or a substituted or non-substituted aralkyl group.)

Specific examples of the nitrogen-containing compound are as follows. Examples of the nitrogen-containing compound (12A) include monoalkylamines such as n-hexylamine; dialkylamines such as di-n-butylamine; trialkylamines such as triethylamine and tri-n-pentylamine; and aromatic amines such as aniline and 2,6-diisopropylaniline.

Examples of the nitrogen-containing compound (12B) include ethylenediamine and N,N,N′,N′-tetramethylethylenediamine.

Examples of the nitrogen-containing compound (12C) include polyamine compounds such as polyethyleneimine and polyallylamine; and polymers such as dimethylaminoethylacrylamide.

Examples of the amido group-containing compound include formamide, N-methylformamide, N,N-dimethylformamide, acetamide, N-methylacetamide, N,N-dimethylacetamide, propionamide, benzamide, pyrrolidone, and N-methylpyrrolidone.

Examples of the urea compound include urea, methylurea, 1,1-dimethylurea, 1,3-dimethylurea, 1,1,3,3-tetramethylurea, 1,3-diphenylurea, and tributylthiourea.

Examples of the nitrogen-containing heterocyclic compound include pyridines such as pyridine and 2-methylpyridine; morpholines such as N-propylmorpholine and N-(undecan-1-yl-carbonyloxyethyl)morpholine; pyrazine; and pyrazole.

Examples of the nitrogen-containing compound having an acid-releasable group include N-t-butoxycarbonylpiperidine, N-t-butoxycarbonylimidazole, N-t-butoxycarbonylbenzimidazole, N-t-butoxycarbonyl-2-phenylbenzimidazole, N-(t-butoxycarbonyl)di-n-octylamine, N-(t-butoxycarbonyl)diethanolamine, N-(t-butoxycarbonyl)dicyclohexylamine, N-(t-butoxycarbonyl)diphenylamine, N-t-butoxycarbonyl-4-hydroxypiperidine, and N-t-amyloxycarbonyl-4-hydroxypiperidine.

Among such compounds, the nitrogen-containing compound serving as acid diffusion control agent [C] is preferably at least one species selected from the group consisting of the nitrogen-containing compound (12A) and the nitrogen-containing heterocyclic compound; more preferably at least one species selected from the group consisting of trialkylamines, aromatic amines, and morpholines; still more preferably at least one species selected from the group consisting of tri-n-pentylamine, 2,6-diisopropylaniline, and N-(undecan-1-yl-carbonyloxyethyl)morpholine.

### Light-Degradable Base

The light-degradable base is a compound which generates an acid through radiation, but the acid does not cause or hardly causes dissociation of an acid-releasable group under conditions in use thereof. Examples of the light-degradable base include compounds which generate an acid weaker than the acid generated by acid generator [B] via light exposure. Among such compounds, the light-degradable base which may be suitably employed is an onium salt which can generate carboxylic acid, sulfonic acid, or sulfonamide through radiation.

Specific examples of preferred light-degradable bases include onium salts having a carboxylate anion structure. Specific examples thereof include onium salt compounds represented by formula (13).

R⁶¹—COO⁻Z⁺ (13)

(In formula (13), R⁶¹ represents a C1 to C30 monovalent organic group; and Z⁺ represents a monovalent cation.)

In formula (13), examples of the C1 to C30 monovalent organic group represented by R⁶¹ include a C1 to C30 monovalent hydrocarbon group, C1 to C30 monovalent group b having a divalent heteroatom-containing moiety between carbon atoms of a hydrocarbon group or at the terminal on the chemical bond side, and a monovalent group in which at least one hydrogen atom of a hydrocarbon group or monovalent group b is substituted by a heteroatom-containing monovalent group. The C1 to C30 monovalent organic group represented by R⁶¹ preferably contains an aromatic ring structure. The aromatic ring structure may have a structure in which a part of or the entire hydrogen atoms of an aromatic ring is substituted. Examples of the substituent which substitutes the hydrogen atoms of the aromatic ring include an iodine atom, a hydroxy group, a trifluoromethyl group, and a monovalent group having a benzene ring substituted by at least one iodine atom.

The cation represented by Z⁺ is preferably an organic cation, particularly preferably a radiation-sensitive onium cation. From the viewpoint of enhancement in lithographic characteristics of the present composition, particularly, the cation is preferably a sulfonium cation or an iodonium cation, for example, a cation represented by formula (7), (8), or (9).

Specific examples of the light-degradable base include compounds represented by the following formulas. However, the light-degradable base is not limited to the following structures.

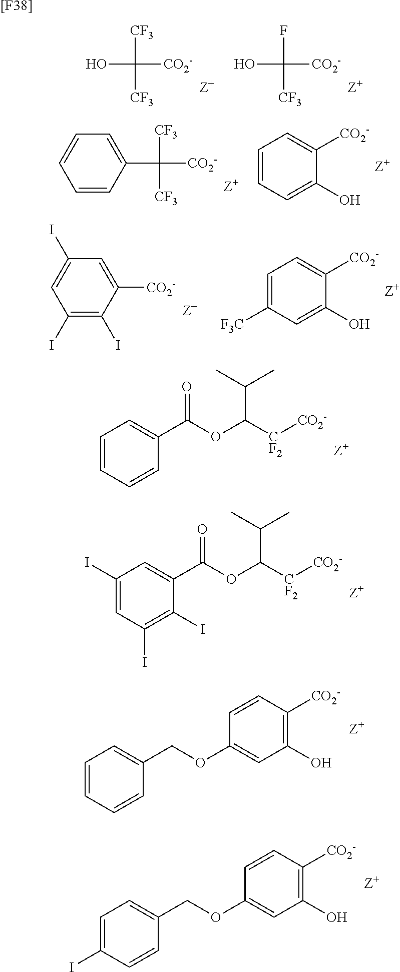

(In the above formulas, Z⁺ represents a cation represented by formula (7), (8), or (9).)

When the present composition contains acid diffusion control agent [C], the relative amount of acid diffusion control agent [C] in the present composition with respect to 100 parts by mass of polymer [A] is preferably 0.1 mass % or more, more preferably 1 mass % or more, still more preferably 3 mass % or more. Also, the relative amount of acid diffusion control agent [C] in the present composition with respect to 100 parts by mass of polymer [A] is preferably 20 mass % or less, more preferably 15 mass % or less, still more preferably 10 mass % or less. Adjusting the acid diffusion control agent [C] content to satisfy the above conditions is preferred, since LWR performance of the present composition can be further enhanced. Acid diffusion control agent [C] may be used singly or in combination of two or more species.

### <[D] Solvent>

No particular limitation is imposed on solvent [D], so long as the solvent can dissolve or disperse components included in the present composition. Examples of solvent [D] include an alcohol, an ether, a ketone, an amide, an ester, and a hydrocarbon.

Examples of the alcohol include C1 to C18 aliphatic monohydric alcohols such as 4-methyl-2-pentanol and n-hexanol; C3 to C18 alicyclic monohydric alcohols such as cyclohexanol; C2 to C18 polyhydric alcohols such as 1,2-propylene glycol; and C3 to C19 polyhydric alcohol partial ethers such as propylene glycol monomethyl ether. Examples of the ether include dialkyl ethers such as diethyl ether, dipropyl ether, dibutyl ether, dipentyl ether, diisoamyl ether, dihexyl ether, and diheptyl ether; cyclic ethers such as tetrahydrofuran and tetrahydropyran; and aromatic ring-containing ethers such as diphenyl ether and anisole.

Examples of the ketone include chain ketones such as acetone, methyl ethyl ketone, methyl n-propyl ketone, methyl n-butyl ketone, diethyl ketone, methyl iso-butyl ketone, 2-heptanone, ethyl n-butyl ketone, methyl n-hexyl ketone, diisobutyl ketone, and trimethylnonanone; cyclic ketones such as cyclopentanone, cyclohexanone, cycloheptanone, cyclooctanone, and methylcyclohexanone; 2,4-pentanedione, acetonylacetone; acetophenone; and diaceton alcohol. Examples of the amide include cyclic amides such as N,N′-dimethylimidazolidinone and N-methylpyrrolidone; and chain amides such as N-methylformamide, N,N-dimethylformamide, N,N-diethylformamide, acetamide, N-methylacetamide, N,N-dimethylacetamide, and N-methylpropionamide.

Examples of the ester include monocarboxylic acid esters such as n-butyl acetate and ethyl lactate; polyhydric alcohol carboxylates such as propylene glycol acetate; polyhydric alcohol partial ether carboxylates such as propylene glycol monomethyl ether acetate; polybasic carboxylic acid diesters such as diethyl oxalate; carbonates such as dimethyl carbonate and diethyl carbonate; and cyclic esters such as γ-butyrolactone. Examples of the hydrocarbon include C5 to C12 aliphatic hydrocarbons such as n-pentane and n-hexane; and C6 to C16 aromatic hydrocarbons such as toluene and xylene.

Particularly, solvent [D] preferably includes at least one species selected from the group consisting of the esters and ketones, more preferably at least one species selected from the group consisting of the polyhydric alcohol partial ether carboxylates and cyclic ketones, still more preferably at least one of propylene glycol monomethyl ether acetate, ethyl lactate, and cyclohexanone. Solvent [D] may be used singly or in combination of two or more species.

### <[E] Acid-Releasable Group-Containing Polymer>

Acid-releasable group-containing polymer [E](hereinafter may also be referred to simply as “polymer [E]”) is a polymer which has an acid-releasable group and no structural unit (I). In the present composition, at least one polymer species selected from the group consisting of polymer [A] and a polymer differing from polymer [A] preferably contains the structural unit (II) having an acid-releasable group. Notably, polymer [E] differs from polymer [A]. When a polymer component of the present composition contains the structural unit (II), an acid-releasable group is released by an acid generated by exposing the present composition to light, to thereby form an acidic group, whereby the solubility of the polymer component in a developer can be modified. As a result, the present composition can be provided with suitable lithographic characteristics.

Examples of the structural unit (II) included in polymer [E] include structural units as described in relation to those which polymer [A] may contain as the structural unit (II). Also, polymer [E] may contain at least one structural unit as described in relation to those which polymer [A] may contain as any of the structural units (III) to (VII). Preferred ranges of the relative amount of each of the structural units (II) to (VII) and those of the weight average molecular weight and molecular weight distribution of polymer [E] are the same as those in relation to polymer [A].

The polymer [E] content of the present composition based on 100 parts by mass of polymer [A] is preferably 20 parts by mass or less, more preferably 10 parts by mass or less, still more preferably 5 parts by mass or less. Notably, the present composition may contain polymer [E]singly or in combination of two or more species.

### <[F] High-Fluorine Content Polymer>

High-fluorine content polymer [F] (hereinafter may also be referred to simply as “polymer [F]”) is a polymer having a fluorine atom content (by mass) greater than that of polymer [A]. Polymer [F] is incorporated into the present composition as, for example, a water-repellent additive.

No particular limitation is imposed on the fluorine atom content of polymer [F], so long as it is greater than the fluorine atom content of polymer [A]. The fluorine atom content of polymer [F] is preferably 1 mass % or more, more preferably 2 mass % or more, still more preferably 4 mass % or more, particularly preferably 7 mass % or more. Also, the fluorine atom content of polymer [F] is preferably 60 mass % or less, more preferably 40 mass % or less, still more preferably 30 mass % or less. The fluorine atom content (mass %) of a polymer can be obtained by determining the structure of the polymer through ¹³C-NMR spectrometry or the like and calculating the content based on the structure determined.

Examples of the structural unit contained in polymer [F] include the below-described structural units (Fa) and (Fb). Polymer [F] may contain structural units (Fa) and (Fb), respectively, singly or in combination of two or more species.

### [Structural Unit (Fa)]

The structural unit (Fa) is a structural unit represented by formula (14a). The fluorine atom content of polymer [F] may be tuned by incorporating the structural unit (Fa).

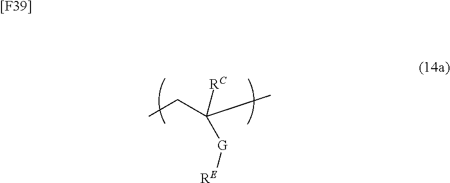

(In formula (14a), R^{C} represents a hydrogen atom, a fluoro group, a methyl group, or a trifluoromethyl group; G represents a single bond, an oxygen atom, a sulfur atom, —CO—O—, —SO₂—O—NH—, —CO—NH—, or —O—CO—NH—; and R^{E} represents a C1 to C6 monovalent fluorinated chain hydrocarbon group or a C4 to C20 monovalent fluorinated alicyclic hydrocarbon group.)

Examples of the C1 to C6 monovalent fluorinated chain hydrocarbon group represented by R^{E} include a trifluoromethyl group, a 2,2,2-trifluoroethyl group, a perfluoroethyl group, a 2,2,3,3,3-pentafluoropropyl group, a 1,1,1,3,3,3-hexafluoropropyl group, a perfluoro-n-propyl group, a perfluoroisopropyl group, a perfluoro-n-butyl group, a perfluoroisobutyl group, a perfluoro-t-butyl group, a 2,2,3,3,4,4,5,5-octafluoropentyl group, and a perfluorohexyl group.

Examples of the C4 to C20 monovalent fluorinated alicyclic hydrocarbon group represented by R^{E} include a monofluorocyclopentyl group, a difluorocyclopentyl group, a perfluorocyclopentyl group, a monofluorocyclohexyl group, a difluorocyclohexyl group, a perfluorocyclohexylmethyl group, a fluoronorbornyl group, a fluoroadamantyl group, a fluorobornyl group, a fluoroisobornyl group, a fluorotricyclodecyl group, and a fluorotetracyclodecyl group.

Examples of the monomer which can provide the structural unit (Fa) include a (meth)acrylate ester having a fluorinated chain hydrocarbon group, and a (meth)acrylate ester having a fluorinated alicyclic hydrocarbon group. Specific examples of the (meth)acrylate ester having a fluorinated chain hydrocarbon group include linear-chain segment-fluorinated alkyl (meth)acrylate esters such as 2,2,2-trifluoroethyl (meth)acrylate; branched-chain segment-fluorinated alkyl (meth)acrylate esters such as 1,1,1,3,3,3-hexafluoroisopropyl (meth)acrylate; linear-chain perfluoroalkyl (meth)acrylate esters such as perfluoroethyl (meth)acrylate; and branched-chain perfluoroalkyl (meth)acrylate esters such as perfluoroisopropyl (meth)acrylate ester.

Examples of the (meth)acrylate ester having a fluorinated alicyclic hydrocarbon group include (meth)acrylate esters having a monocyclic fluorinated alicyclic saturated hydrocarbon group such as perfluorocyclohexylmethyl (meth)acrylate, monofluorocyclopentyl (meth)acrylate ester, and perfluorocyclopentyl (meth)acrylate ester; and (meth)acrylate esters having a polycyclic fluorinated alicyclic saturated hydrocarbon group such as fluoronorbornyl (meth)acrylate ester.

When polymer [F] contains the structural unit (Fa), the relative amount of structural unit (Fa) with respect to all the structural units forming polymer [F] is preferably 5 mol % or more, more preferably 10 mol % or more, still more preferably 20 mol % or more.

### [Structural Unit (Fb)]

The structural unit (Fb) is a structural unit represented by formula (14b). Polymer [F], containing the structural unit (Fb), exhibits enhanced hydrophobicity, whereby the dynamic contact angle on a resist film formed from the present composition can be further enhanced.

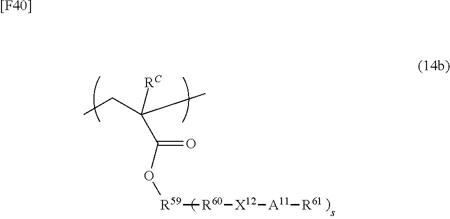

(In formula (14b), R^{F} represents a hydrogen atom, a fluoro group, a methyl group, or a trifluoromethyl group; R⁵⁹ represents an (s+1)-valent C1 to C20 hydrocarbon group, or a group formed of the hydrocarbon group to which an oxygen atom, a sulfur atom, —NR′—, a carbonyl group, —CO—O—, or —CO—NH— is bonded on the R⁶⁰ side terminal; R′ represents a hydrogen atom or a monovalent organic group; R⁶⁰ represents a single bond, a C1 to C10 divalent chain hydrocarbon group, or a C4 to C20 divalent alicyclic hydrocarbon group; X¹² represents a C1 to C20 divalent fluorinated chain hydrocarbon group; A¹¹ represents an oxygen atom, —NR″—, —CO—O—*, or —SO₂—O—*; R″ represents a hydrogen atom or a monovalent organic group; * represents a bonding site to R⁶¹; R⁶¹ represents a hydrogen atom or a monovalent organic group; s is an integer of 1 to 3; when s is 2 or 3, a plurality of R⁶⁰, X¹², A¹¹, and R⁶¹ are respectively identical to or different from one another.)

When R⁶¹ is a hydrogen atom, the solubility of polymer [F] in an alkaline developer can be enhanced, which is preferred. Examples of the monovalent organic group represented by R⁶¹ include a C1 to C30 hydrocarbon group which may have an acid-releasable group, an alkali-releasable group, or a substituent.

When polymer [F] contains the structural unit (Fb), the relative amount of structural unit (Fb) in all the structural units forming polymer [F] is preferably 5 mol % or more, more preferably 10 mol % or more, still more preferably 20 mol % or more.

In addition to the structural units (Fa) and (Fb), polymer [F] may further contain a structural unit which has an acid-releasable group and which differs from the structural units (Fa) and (Fb) (hereinafter may also be referred to as a “structural unit (Fc)”). When polymer [F] contains the structural unit (Fc), the obtained resist pattern becomes suitable. Examples of the structural unit (Fc) include structural units (II) and the like, which are described in relation to polymer [A].

When polymer [F] contains the structural unit (Fc), the relative amount of structural unit (Fc) in all the structural units forming polymer [F] is preferably 5 mol % or more, more preferably 25 mol % or more, still more preferably 50 mol % or more. Also, the structural unit (Fc) content with respect to all the structural units forming polymer [F] is preferably 90 mol % or less, more preferably 80 mol % or less, still more preferably 70 mol % or less.

The Mw of polymer [F] determined through GPC is preferably 1,000 or more, more preferably 3,000 or more, still more preferably 4,000 or more. Also, the Mw of polymer [F] is preferably 50,000 or less, more preferably 30,000 or less, still more preferably 20,000 or less. The molecular weight distribution (Mw/Mn) which is represented by a ratio of Mw of polymer [F] to Mn of polymer [F] determined through GPC is generally 1 or more, preferably 1.2 or more. Also, the Mw/Mn of polymer [F] is preferably 5 or less, more preferably 3 or less.

When the present composition contains polymer [F], the relative amount of polymer [F] in the present composition with respect to 100 parts by mass of polymer [A] is preferably 0.05 parts by mass or more, more preferably 0.1 parts by mass or more, still more preferably 0.5 parts by mass or more. Also, the polymer [F] content with respect to 100 parts by mass of polymer [A] is preferably 10 parts by mass or less, more preferably 5 parts by mass or less, still more preferably 3 parts by mass or less. Notably, the present composition may contain polymer [F] singly or in combination of two or more species.

### <Additional and Optional Component>

The present composition may further contain a component which differs from the aforementioned polymer [A], acid generator [B], acid diffusion control agent [C], solvent [D], acid-releasable group-containing polymer [E], and high-fluorine content polymer [F] (hereinafter the component may also be referred to as “additional and optional component”). Examples of the additional and optional component include a surfactant, a compound having an alicyclic skeleton (e.g., 1-adamantanecarboxylic acid, 2-adamantanone, or t-butyl deoxycholate), a sensitizer, and a localization accelerator. So long as the effect of the present disclosure are not impaired, the additional and optional component content of the present composition may be appropriately set depending on the property of the component.

### <Method of Producing Radiation-Sensitive Composition>

The present composition may be produced through, for example, the following procedure: mixing polymer [A] with an optional component such as solvent [D] at a desired ratio and filtering the resultant mixture preferably by means of a filter (e.g., a filter having a pore size of about 0.2 μm) or the like. The solid content of the present composition is preferably 0.1 mass % or more, more preferably 0.5 mass % or more, still more preferably 1 mass % or more. Also, the solid content of the present composition is preferably 50 mass % or less, more preferably 20 mass % or less, still more preferably 5 mass % or less. Adjusting the solid content of the present composition to satisfy the above conditions is preferred, since coatability of the composition can be enhanced, to thereby obtain a resist pattern having a suitable shape.

The thus-obtained present composition may also be used as a composition for forming a positive pattern, which is employed for pattern formation by use of an alkaline developer. Alternatively, the present composition may be used as a composition for forming a negative pattern by use of a developer containing organic solvent.

### <Method of Forming Resist Pattern>

The resist pattern formation method of the present disclosure includes a step of applying the present composition on one surface of a substrate (hereinafter may also be referred to as a “application step”), a step of exposing to light a resist film obtained in the application step (hereinafter may also be referred to as a “light-exposure step”), and a step of developing the light-exposed resist film (hereinafter may also be referred to as a “development step”). Examples of the pattern obtained through the resist pattern formation method of the present disclosure include a line-and-space pattern and a hole pattern. Since a resist film is formed by use of the present composition in the resist pattern formation method of the present disclosure, a resist pattern which exhibits excellent sensitivity and lithographic characteristics and which has few development faults can be formed. Particularly, since the present composition has wide process windows, occurrence of faults which would otherwise be caused by variation in process conditions can be suppressed, through the resist pattern formation method of the disclosure for forming a resist film by use of the present composition. The steps will next be described in detail.

### [Application Step]

In this step, the present composition is applied onto one surface of a substrate, to thereby form a resist film on the substrate. A conventionally known substrate can be used as a substrate on which resist film is to be formed. Examples of the substrate include a silicon wafer and a wafer coated with silicon dioxide or aluminum. For example, an organic or inorganic anti-reflection film disclosed in, for example, Japanese Patent Publication (kokoku) No. 1994-12452 or Japanese Patent Application laid-Open (kokai) No. 1984-93448 may be formed on a substrate to be used. Examples of the method of applying the present composition include spin coating, flow casting, and roller coating. After application, the applied composition may be subjected to soft baking (SB) so as to evaporate the solvent remaining in the coating film. The temperature of SB is preferably 60° C. or higher, more preferably 80° C. or higher. Also, the temperature of SB is preferably 140° C. or lower, more preferably 120° C. or lower. The time of SB is preferably 5 seconds or longer, more preferably 10 seconds or longer, and preferably 600 seconds or shorter, more preferably 300 seconds or shorter. The average thickness of the formed resist film is preferably 10 to 1,000 nm, more preferably 20 to 500 nm.

### [Light-Exposure Step]

In this step, the resist film formed through the above application step is exposed to light. In the light exposure, the resist film is irradiated with radiation by the mediation of a photomask or, in some cases, a liquid immersion medium such as water. The radiation is selected in accordance with the line width of a target pattern, and examples thereof include electromagnetic waves such as visible light, a near-UV ray, a far-UV ray, an extreme UV (EUV) ray, an X-ray, and a γ-ray; and charged particle rays such as an electron beam and an α-ray. Among them, the radiation applied to the resist film formed from the present composition is preferably a far-UV ray, an EUV ray, or an electron beam, more preferably ArF excimer laser light (wavelength: 193 nm), KrF excimer laser light (wavelength: 248 nm), an EUV ray, or an electron beam, still more preferably ArF excimer laser light, an EUV ray, or an electron beam, yet more preferably an EUV ray or an electron beam, particularly preferably an EUV ray.

After completion of the above light exposure, post exposure baking (PEB) is preferably performed so as to accelerate dissociation of an acid-releasable group of polymer [A] and other components in the light-exposed part of the resist film. Through PEB, the difference in dissolution performance with respect to a developer between the exposed part and the unexposed part can be increased. The temperature at PEB is preferably 50° C. or higher, more preferably 70° C. or higher, and preferably 180° C. or lower, more preferably 130° C. or lower. The time of PEB is preferably 5 seconds or longer, more preferably 10 seconds or longer, and preferably 600 seconds or shorter, more preferably 300 seconds or shorter.

### [Development Step]

In this step, the resist film which has been exposed to light in the above step is developed, whereby a resist pattern of interest can be formed. Generally, after development, the developed film is washed with a rinse liquid (e.g., water or alcohol) and then dried. The development method employed in the development step may be development with alkali or with organic solvent.

Examples of the developer employed in the alkali development include aqueous alkaline solutions in which at least one species from among alkaline compounds such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethylammonium hydroxide (TMAH), pyrrole, piperidine, choline, 1,8-diazabicyclo[5.4.0]-7-undecene, 1,5-diazabicyclo[4.3.0]-5-nonene, and the like is dissolved. Among such alkaline solutions, an aqueous TMAH solution is preferred, with 2.38-mass % aqueous TMAH solution being more preferred.

In the case of development with an organic solvent, examples of the developer include of organic solvents such as hydrocarbnons, ethers, esters, ketones, and alcohols; and one or more of solvents each containing any of the above organic solvents. Examples of the organic solvent employed as the developer include the solvents as exemplified in relation to solvent [D] of the present composition. Among them, esters and ketones are preferred. Among the esters, acetate esters are preferred, with n-butyl acetate being more preferred. Among the ketones, chain ketones are preferred, with 2-heptanone being more preferred. The organic solvent content of the developer is preferably 80 mass % or more, more preferably 90 mass % or more, still more preferably 95 mass % or more, particularly preferably 99 mass % or more. Examples of developer components other than organic solvent include water and silicone oil.

Examples of the development method include a dipping method (i.e., dipping a substrate in a bath filled with a developer for a specific time); a paddle method (i.e., putting a developer in a substrate to form a drop by surface tension and allowing the substrate to stand for a specific time); a spray method (i.e., spraying a developer onto a substrate); and a dynamic dispense method (i.e., continuously jetting a developer at a specific speed to a substrate rotating at a specific speed through a developer jetting nozzle with scanning).

The present disclosure described hereinabove provides the following means.

[1] A radiation-sensitive composition comprising a polymer which contains a structural unit (I) represented by formula (1).

[2] A radiation-sensitive composition as described in [1] above, wherein the structural unit (I) is at least one species selected from the group consisting of a structural unit derived from a compound represented by formula (4) and a structural unit derived from a compound represented by formula (5).

[3] A radiation-sensitive composition as described in [1] or [2] above, wherein at least one species selected from the group consisting of the polymer containing the structural unit (I) and a polymer differing from the polymer containing the structural unit (I) contains a structural unit (II) having an acid-releasable group.

[4] A radiation-sensitive composition as described in any of [1] to [3] above, wherein the polymer containing structural unit (I) further contains a structural unit (III) having a hydroxy group bonded to an aromatic ring.

[5] A resist pattern formation method, comprising a step of forming a resist film on a substrate by use of a radiation-sensitive composition as recited in any of [1] to [4] above, a step of exposing the resist film to light, and a step of developing the light-exposed resist film.

[6] A polymer containing a structural unit represented by formula (1).

[7] A compound represented by formula (4).

[8] A compound represented by formula (5).

### EXAMPLES

The present disclosure will next be described in detail by way of examples, which should not be construed as limiting the disclosure thereto. Unless otherwise specified, the units “part(s)” and “%” in the Examples are on a mass basis. Measurements in the Examples and Comparative Examples were conducted by the following procedures.

### [Weight Average Molecular Weight (Mw) and Number Average Molecular Weight (Mn)]

The molecular weights were determined through gel permeation chromatography (GPC) with GPC columns (G2000HXL×2, G3000HXL×1, and G4000HXL×1) (products of Tosoh Corp.). The analytical conditions were as follows: flow rate: 1.0 mL/min, elution solvent: tetrahydrofuran, sample concentration: 1.0 mass %, sample injection: 100 μL, column temperature: 40° C., detector: differential refractometer, and standard: monodispersed polystyrene. Also, polydispersity index (Mw/Mn) was calculated by measurements of Mw and Mn.

### [¹³C-NMR Analysis]

Each structural unit content (mol %) of a polymer was determined by means of an NMR (JNM-ECX400, product of JEOL Ltd.) with DMSO-d₆ as a solvent for measurement.

### <Synthesis of Compounds>

### [Synthesis Example 1] Synthesis of Compound (M-1)

4-Vinylbenzaldehyde (12 g), triphenylsulfonium-α,α,β,β-tetrafluoro-2-(5,6-dihydroxybicyclo[2.2.1]heptan-2-yl)ethane sulfonate (40 g), trimethyl orthoformate (9 g), and p-toluenesulfonic acid monohydrate (2.7 g) were dissolved in dichloromethane (240 g), and the solution was caused to react at room temperature for 5 hours. After completion of reaction, the reaction mixture was washed five times with water (200 g), and dichloromethane was distilled out, to thereby yield 64 g of a crude product. The crude product was then purified with a column, to thereby yield 50 g of compound (M-1) with a purity of 99.3% as determined through HPLC.

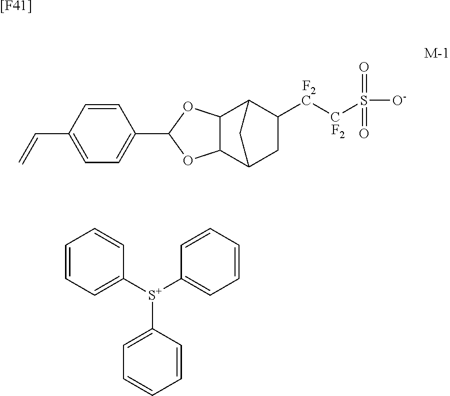

### <Synthesis Polymers>

The following monomers were used to form polymers.

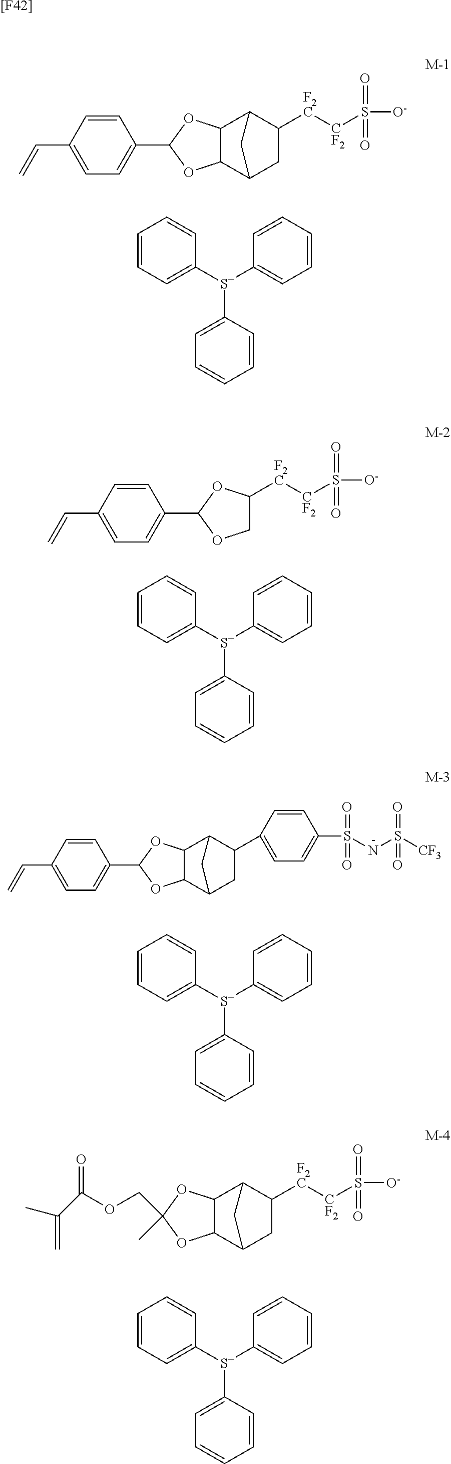

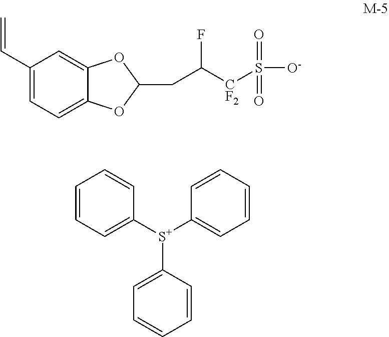 [SMILES: C=Cc1ccc2c(c1)OC(CC(F)C(F)(F)S(=O)(=O)[O-])O2 ; c1ccc([S+](c2ccccc2)c2ccccc2)cc1]

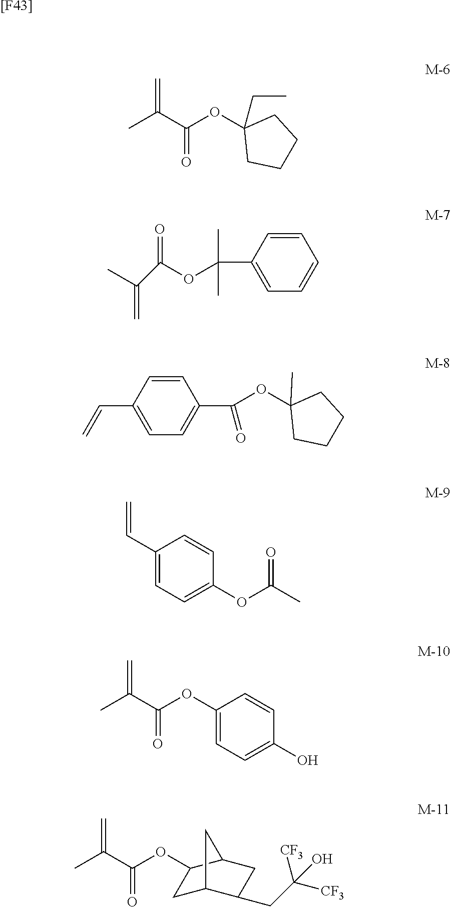

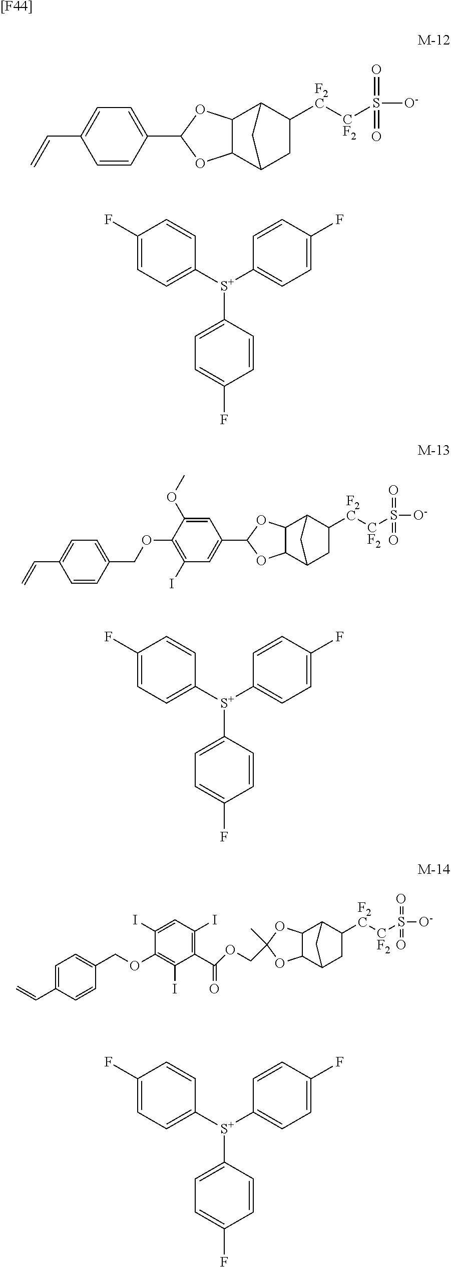

### [Synthesis Example 1] (Synthesis of Polymer (A-1))

Compound (M-1) (15 mol %), compound (M-6) (55 mol %), compound (M-9) (30 mol %), and 2,2′-azobis(methyl isobutyrate) (12 mol % with respect to all monomers) as a polymerization initiator were dissolved in propylene glycol monomethyl ether (40 g), to thereby prepare a monomer solution. Separately, propylene glycol monomethyl ether (20 g) was added to a 100-mL three-neck flask. The atmosphere of the flask was purged with nitrogen for 30 minutes, and the contents were heated at 85° C. under stirring. Subsequently, the above-prepared monomer solution was added dropwise to the flask over 3 hours by means of a dropping funnel. After addition, the mixture was further stirred at 85° C. for 3 hours. After completion of the polymerization reaction, ethyl acetate (56 g), methanol (24 g), water (6.4 g), and hexane (200 g) were added to the polymerization solution with mixing, and the mixture was transferred to a 1L separating funnel. The contents were allowed to stand for 30 minutes, and the lower layer was recovered. The solvent of the recovered matter was substituted by propylene glycol monomethyl ether, to thereby yield 80 g of a solution. Next, methanol (100 g), triethylamine (10 g), and water (2 g) were added thereto. While the resultant mixture was refluxed at a boiling temperature, hydrolysis was performed for 6 hours. After completion of hydrolysis reaction, the solvent and triethylamine were distilled off under reduced pressure. The thus-obtained polymer was dissolved in propylene glycol monomethyl ether, to thereby yield polymer (A-1) solution having a solid content of 15%. The polymer (A-1) was found to have an Mw of 8,200 and an Mw/Mn of 1.6. The ¹³C-NMR analysis have revealed that the relative amount of a structural unit derived from compound (M-1), the relative amount of a structural unit derived from compound (M-6), and the p-hydroxystyrene unit derived from compound (M-9) were 16.2 mol %, 53.3 mol %, and 30.5 mol %, respectively.

### [Synthesis Examples 2 to 8, and 10 to 13] (Synthesis of Polymers (A-2) to (A-8), and (A-10) to (A-13))

The procedure of Synthesis Example 1 was repeated, except that appropriate monomers were employed, to thereby yield 15%-solid content polymer solutions of polymers (A-2) to (A-8), and (A-10) to (A-13), respectively. Table 1 shows the types and amounts of monomers employed. Also, Table 2 shows the structural unit contents of the obtained polymers.

### [Synthesis Example 9] (Synthesis of Polymer (A-9))

Compound (M-1) (15 mol %), compound (M-6) (55 mol %), compound (M-10) (30 mol %), and 2,2′-azobis(methyl isobutyrate) (12 mol % with respect to all monomers) as a polymerization initiator were dissolved in propylene glycol monomethyl ether (40 g), to thereby prepare a monomer solution. Separately, propylene glycol monomethyl ether (20 g) was added to a 500-mL three-neck flask. The atmosphere of the flask was purged with nitrogen for 30 minutes, and the contents were heated at 85° C. under stirring. Subsequently, the above-prepared monomer solution was added dropwise to the flask over 3 hours by means of a dropping funnel. After addition, the mixture was further stirred at 85° C. for 3 hours. After completion of the polymerization reaction, ethyl acetate (56 g), methanol (24 g), water (6.4 g), and hexane (200 g) were added to the polymerization solution with mixing, and the mixture was transferred to a 1L separating funnel. The contents were allowed to stand for 30 minutes, and the lower layer was recovered. The recovered matter was dissolved in propylene glycol monomethyl ether acetate, to thereby yield polymer (A-9) solution having a solid content of 15%. The polymer (A-9) was found to have an Mw of 6,700 and an Mw/Mn of 1.5. The ¹³C-NMR analysis have revealed that the relative amounts of a structural unit derived from compound (M-1), (M-6), and (M-9) were found to be 15.7 mol %, 53.2 mol %, and 31.1 mol %, respectively.

TABLE 1

| Synthesis | | Amount of Monomer (mol %) | | | | | | | |
|---|---|---|---|---|---|---|---|---|---|
| Examples | Polymer | Monomer 1 | | Monomer 2 | | Monomer 3 | | Monomer 4 | |
| Synthesis | A-1 | M-1 | 15 | M-6 | 55 | M-9 | 30 | — | — |
| Example 1 | | | | | | | | | |
| Synthesis | A-2 | M-2 | 15 | M-6 | 55 | M-9 | 30 | — | — |
| Example 2 | | | | | | | | | |
| Synthesis | A-3 | M-3 | 15 | M-6 | 55 | M-9 | 30 | — | — |
| Example 3 | | | | | | | | | |
| Synthesis | A-4 | M-4 | 15 | M-6 | 55 | M-9 | 30 | — | — |
| Example 4 | | | | | | | | | |
| Synthesis | A-5 | M-5 | 15 | M-6 | 55 | M-9 | 30 | — | — |
| Example 5 | | | | | | | | | |
| Synthesis | A-6 | M-1 | 15 | M-7 | 55 | M-9 | 30 | — | — |
| Example 6 | | | | | | | | | |
| Synthesis | A-7 | M-1 | 15 | M-8 | 55 | M-9 | 30 | — | — |
| Example 7 | | | | | | | | | |
| Synthesis | A-8 | M-1 | 15 | M-6 | 45 | M-9 | 30 | M-11 | 10 |
| Example 8 | | | | | | | | | |
| Synthesis | A-9 | M-1 | 15 | M-6 | 55 | M-10 | 30 | — | — |
| Example 9 | | | | | | | | | |
| Synthesis | A-10 | — | — | M-6 | 65 | M-9 | 35 | — | — |
| Example 10 | | | | | | | | | |
| Synthesis | A-11 | M-12 | 15 | M-6 | 55 | M-9 | 30 | — | — |
| Example 11 | | | | | | | | | |
| Synthesis | A-12 | M-13 | 15 | M-6 | 55 | M-9 | 30 | — | — |
| Example 12 | | | | | | | | | |
| Synthesis | A-13 | M-14 | 15 | M-6 | 55 | M-9 | 30 | — | — |
| Example 13 | | | | | | | | | |

TABLE 2

| Synthesis | | Polymer Composition (mol %) | | | | | | | | | |
|---|---|---|---|---|---|---|---|---|---|---|---|
| Examples | Polymer | Monomer 1 | | Monomer 2 | | Monomer 3 | | Monomer 4 | | Mw | Mw/Mn |
| Synthesis | A-1 | M-1 | 16.2 | M-6 | 53.3 | M-9 | 30.5 | — | — | 8200 | 1.6 |
| Example 1 | | | | | | | | | | | |
| Synthesis | A-2 | M-2 | 16.3 | M-6 | 52.5 | M-9 | 31.2 | — | — | 7800 | 1.6 |
| Example 2 | | | | | | | | | | | |
| Synthesis | A-3 | M-3 | 15.1 | M-6 | 54.1 | M-9 | 30.8 | — | — | 8500 | 1.6 |
| Example 3 | | | | | | | | | | | |
| Synthesis | A-4 | M-4 | 13.5 | M-6 | 53.1 | M-9 | 33.4 | — | — | 7800 | 1.6 |
| Example 4 | | | | | | | | | | | |
| Synthesis | A-5 | M-5 | 15.5 | M-6 | 53.1 | M-9 | 31.4 | — | — | 8500 | 1.6 |
| Example 5 | | | | | | | | | | | |
| Synthesis | A-6 | M-1 | 15.5 | M-7 | 53.5 | M-9 | 31.0 | — | — | 8400 | 1.6 |
| Example 6 | | | | | | | | | | | |
| Synthesis | A-7 | M-1 | 16.1 | M-8 | 54.5 | M-9 | 29.4 | — | — | 8600 | 1.7 |
| Example 7 | | | | | | | | | | | |
| Synthesis | A-8 | M-1 | 15.2 | M-6 | 43.5 | M-9 | 31.2 | M-11 | 10.1 | 8000 | 1.6 |
| Example 8 | | | | | | | | | | | |
| Synthesis | A-9 | M-1 | 15.7 | M-6 | 53.2 | M-10 | 31.1 | — | — | 6700 | 1.5 |
| Example 9 | | | | | | | | | | | |
| Synthesis | A-10 | — | — | M-6 | 64.5 | M-9 | 35.5 | — | — | 7900 | 1.6 |
| Example 10 | | | | | | | | | | | |

### <Preparation of Radiation-Sensitive Resin Compositions>

Radiation-sensitive resin compositions of Examples 1 to 20 and Comparative Example 1 were prepared by use of the below-described members of acid generator [B], acid diffusion control agent [C], and solvent [D].

### Acid Generator [B]

PAG-1: a compound represented by formula (PAG-1)

PAG-2: a compound represented by formula (PAG-2)

PAG-3: a compound represented by formula (PAG-3)

PAG-4: a compound represented by formula (PAG-4)

PAG-5: a compound represented by formula (PAG-5)

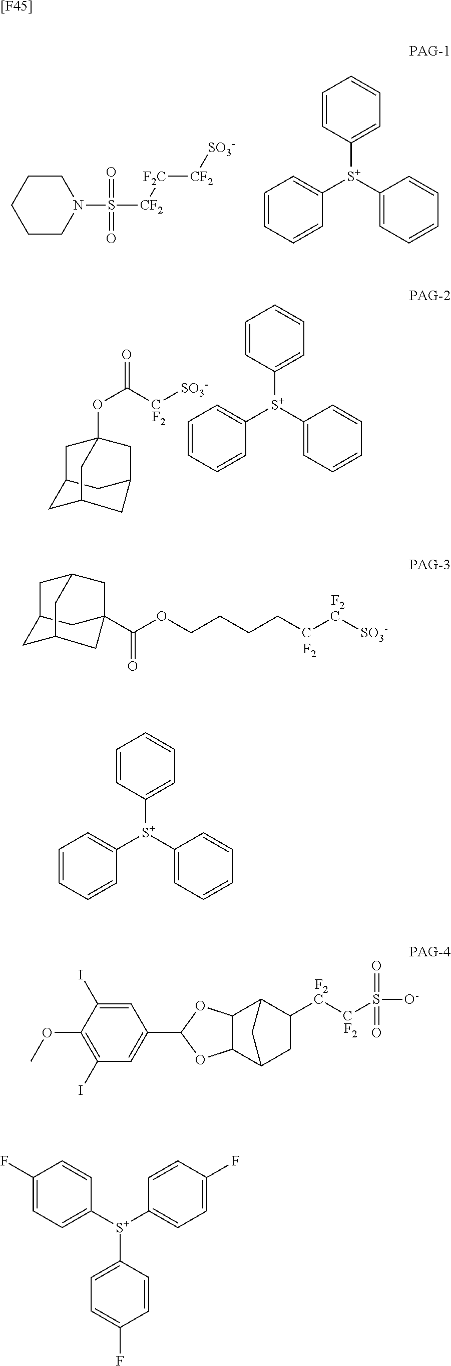

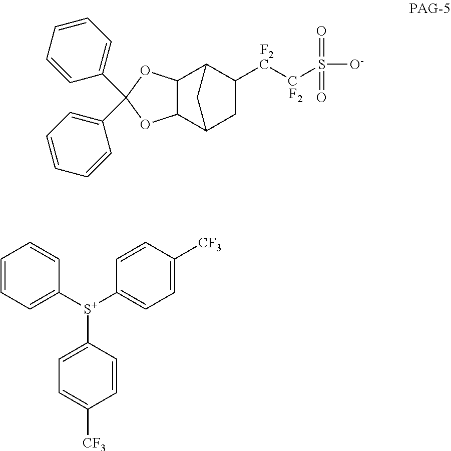

### Acid Diffusion Control Agent [C]

Q-1: a compound represented by formula (Q-1)

Q-2: a compound represented by formula (Q-2)

Q-3: a compound represented by formula (Q-3)

Q-4: a compound represented by formula (Q-4)

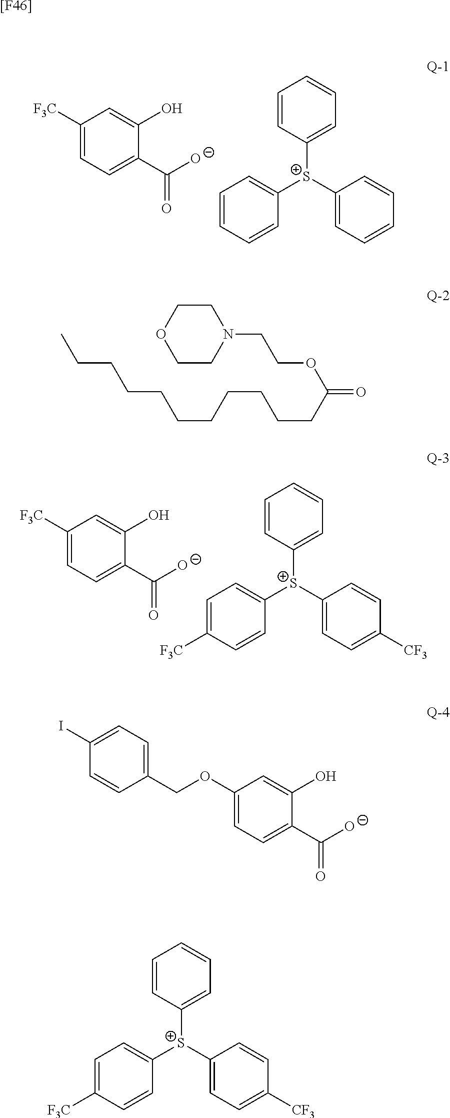

### Solvent [D]

D-1: propylene glycol monomethyl ether acetate

D-2: propylene glycol monomethyl ether

### Example 1

Polymer (A-1) solution (670 parts by mass), acid diffusion control agent (Q-1) (15 parts by mass), solvent (D-1) (1,700 parts by mass), and solvent (D-2) (6,230 parts by mass) were mixed, and the resultant mixture was filtered by means of a membrane filter having a pore size of 0.2 μm, to thereby prepare a radiation-sensitive resin composition (J-1).

### Examples 2 to 20 and Comparative Example 1

The procedure of Example 1 was repeated, except that the types and amounts of the components were changed as shown in Table 3, to thereby prepare radiation-sensitive resin compositions.

TABLE 3

| | Radition- | | | | | Acid Diffusion | | Solvent | |
|---|---|---|---|---|---|---|---|---|---|
| | sensitive | Polymer | | PAG | | Control Agent | | D-1 | D-2 |
| | Resin | | Parts by | | Parts by | | Parts by | (Parts by | (Parts by |
| Examples | Composition | Type | Mass | Type | Mass | Type | Mass | Mass) | Mass) |
| Example 1 | J-1 | (A-1) | 670 | — | 0 | Q-1 | 15 | 1700 | 6230 |
| | | solution | | | | | | | |
| Example 2 | J-2 | (A-2) | 670 | — | 0 | Q-1 | 15 | 1700 | 6230 |
| | | solution | | | | | | | |
| Example 3 | J-3 | (A-3) | 670 | — | 0 | Q-1 | 15 | 1700 | 6230 |
| | | solution | | | | | | | |
| Example 4 | J-4 | (A-4) | 670 | — | 0 | Q-1 | 15 | 1700 | 6230 |
| | | solution | | | | | | | |
| Example 5 | J-5 | (A-5) | 670 | — | 0 | Q-1 | 15 | 1700 | 6230 |
| | | solution | | | | | | | |
| Example 6 | J-6 | (A-6) | 670 | — | 0 | Q-1 | 15 | 1700 | 6230 |
| | | solution | | | | | | | |
| Example 7 | J-7 | (A-7) | 670 | — | 0 | Q-1 | 15 | 1700 | 6230 |
| | | solution | | | | | | | |
| Example 8 | J-8 | (A-8) | 670 | — | 0 | Q-1 | 15 | 1700 | 6230 |
| | | solution | | | | | | | |
| Example 9 | J-9 | (A-9) | 670 | — | 0 | Q-1 | 15 | 1700 | 6230 |
| | | solution | | | | | | | |
| Example 10 | J-10 | (A-1) | 670 | PAG-1 | 10 | Q-1 | 17 | 1900 | 7030 |
| | | solution | | | | | | | |
| Example 11 | J-11 | (A-1) | 670 | PAG-2 | 10 | Q-1 | 17 | 1900 | 7030 |
| | | solution | | | | | | | |
| Example 12 | J-12 | (A-1) | 670 | PAG-3 | 10 | Q-1 | 17 | 1900 | 7030 |
| | | solution | | | | | | | |
| Example 13 | J-13 | (A-1) | 670 | — | 0 | Q-2 | 7 | 1600 | 5830 |
| | | solution | | | | | | | |
| Example 14 | J-15 | (A-11) | 670 | — | 0 | Q-1 | 15 | 1700 | 6230 |
| | | solution | | | | | | | |
| Example 15 | J-16 | (A-12) | 670 | — | 0 | Q-1 | 15 | 1700 | 6230 |
| | | solution | | | | | | | |
| Example 16 | J-17 | (A-13) | 670 | — | 0 | Q-1 | 15 | 1700 | 6230 |
| | | solution | | | | | | | |
| Example 17 | J-18 | (A-11) | 670 | — | 0 | Q-3 | 15 | 1700 | 6230 |
| | | solution | | | | | | | |
| Example 18 | J-19 | (A-11) | 670 | — | 0 | Q-4 | 15 | 1700 | 6230 |
| | | solution | | | | | | | |
| Example 19 | J-20 | (A-11) | 670 | PAG-4 | 10 | Q-3 | 17 | 1900 | 7030 |
| | | solution | | | | | | | |
| Example 20 | J-21 | (A-11) | 670 | PAG-5 | 10 | Q-3 | 17 | 1900 | 7030 |
| | | solution | | | | | | | |
| Comparative | J-14 | (A-10) | 670 | PAG-1 | 25 | Q-1 | 15 | 2000 | 7430 |
| Example 1 | | solution | | | | | | | |

### <Formation of Resist Pattern (EB Exposure and Alkali Development>

A 12-inch silicon wafer having an under-layer film having an average thickness of 60 nm (DUV42, product of Brewer Science, Inc.) was used. Onto the under-layer film, the above-prepared radiation-sensitive resin composition was applied by means of a spin coater (CLEAN TRACK ACT12, product of Tokyo Electron Ltd.). The wafer was subjected to soft baking (SB) at 110° C. for 60 seconds and then cooled at 23° C. for 30 seconds, to thereby form a resist film having an average thickness of 40 nm. Subsequently, the resist film was irradiated with light by means of an EB exposure device (ELS-F150, product of Elionix) through a mask for forming a 20-nm line-and-space pattern at 150 keV and 100 pA. After the light exposure, PEB was performed at 80° C. for 60 seconds. Then, development was performed by use of an alkaline developer (i.e., 2.38 mass % aqueous TMAH) at 23° C. for 30 seconds. The developed product was washed with water and dried, to thereby form a positive-type resist pattern.

### <Assessment>

Each of the above-prepared radiation-sensitive resin compositions was evaluated in terms of sensitivity and process window through the following procedure. The same procedure for forming the resist pattern was repeated. The dimensions of each resist pattern were measured by means of a scanning electron microscope (CG-4100, product of Hitachi High Technology Co., Ltd.). Table 4 shows the results.

### [Sensitivity]

In formation of a resist pattern by use of each of the aforementioned radiation-sensitive resin compositions, the exposure dose for forming 20-nm line-and-space pattern was determined as an optimum exposure dose. The optimum exposure dose was employed as an index of sensitivity (μC/cm², EB sensitivity).

### [Process Window]

By use of a mask for forming a 20-nm line-and-space pattern (1L/1S), various patterns were formed corresponding to exposure doses ranging from a low value to a high value. Generally, application of light of a lower exposure dose results in undesired defects such as bridge formation between patterns, and application of light of a higher exposure dose results in defects such as collapsing of patterns. Accordingly, the range of a resist dimension parameter (i.e., a line width) at which the above defects were not observed was determined. The difference between the maximum and minimum values of the line width was employed as a “Critical dimension (CD) margin.” The greater the CD margin, the wider (more excellent) the process window. In the case where the CD margin was 1.5 nm or more, the score was “good,” and in the case where the CD margin was less than 1.5 nm, the score was “bad.”

TABLE 4

| | Radition- | | | | |
|---|---|---|---|---|---|
| | sensitive | | | EB | |
| | Resin | SB | PEB | Sensitivity | Process |
| Examples | Composition | (° C.) | (° C.) | (μC/cm²) | window |
| Example 1 | J-1 | 110 | 80 | 600 | good |
| Example 2 | J-2 | 110 | 80 | 575 | good |
| Example 3 | J-3 | 110 | 80 | 675 | good |
| Example 4 | J-4 | 110 | 80 | 550 | good |
| Example 5 | J-5 | 110 | 80 | 575 | good |
| Example 6 | J-6 | 110 | 80 | 525 | good |
| Example 7 | J-7 | 110 | 80 | 625 | good |
| Example 8 | J-8 | 110 | 80 | 650 | good |
| Example 9 | J-9 | 110 | 80 | 525 | good |
| Example 10 | J-10 | 110 | 80 | 500 | good |
| Example 11 | J-11 | 110 | 80 | 575 | good |
| Example 12 | J-12 | 110 | 80 | 550 | good |
| Example 13 | J-13 | 110 | 80 | 575 | good |
| Example 14 | J-15 | 110 | 80 | 575 | good |
| Example 15 | J-16 | 110 | 80 | 610 | good |
| Example 16 | J-17 | 110 | 80 | 620 | good |
| Example 17 | J-18 | 110 | 80 | 565 | good |
| Example 18 | J-19 | 110 | 80 | 560 | good |
| Example 19 | J-20 | 110 | 80 | 510 | good |
| Example 20 | J-21 | 110 | 80 | 500 | good |
| Comparative | J-14 | 110 | 80 | 575 | bad |
| Example 1 | | | | | |

As is clear from Table 4, the radiation-sensitive resin compositions of Examples 1 to 20 were found to exhibit high sensitivity and a larger CD margin, as compared with the radiation-sensitive resin composition of Comparative Example 1, indicating a suitably wide process window.

From the test results, when the radiation-sensitive composition of the present disclosure containing a polymer which contains a structural unit (I) represented by formula (1), and the resist pattern formation method employing the radiation-sensitive composition were employed, a high sensitivity to the light exposed, and a resist pattern having a wide process window were found to be attained. Therefore, the radiation-sensitive composition and resist pattern formation method according to the present disclosure are suitable for semiconductor device processing or a similar technique, on which further miniaturization will be imposed.

1: A radiation-sensitive composition comprising a polymer which comprises a structural unit (I) represented by formula (1): 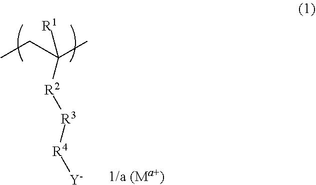
wherein R¹ represents a hydrogen atom, a fluorine atom, a methyl group, or a trifluoromethyl group; R² represents a single bond, a divalent hydrocarbon group, a divalent group F¹ which is formed by substituting any hydrogen atom of a divalent hydrocarbon group with a monovalent substituent, a divalent group F² including —O—, —CO—, —NH—, —COO—, or —CONH— which intervenes between a C—C bond of a divalent hydrocarbon group or the F¹, *¹—COO—R⁷—, or *¹—CONH—R⁷—; R⁷ represents a single bond, a divalent hydrocarbon group, a divalent group F³ which is formed by substituting any hydrogen atom of a divalent hydrocarbon group with a monovalent substituent, or a divalent group F⁴ including —O—, —CO—, —NH—, —COO—, or —CONH— which intervenes between a C—C bond of a divalent hydrocarbon group or the F³; “*¹” represents a site bonding to the carbon atom to which R¹ is bonded; R³ represents a divalent group represented by formula (2) or (3): R⁴ represents a divalent organic group: Y⁻ represents a monovalent anion which can generate a sulfonate group, an imidic acid group, or a methide acid group through exposure to light; and M^{a+} represents an a-valent cation; and a is 1 or 2: 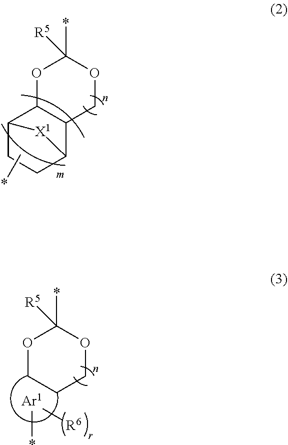
wherein R⁵ represents a hydrogen atom or a monovalent organic group; X¹ represents —CH₂—, —NH—, —O—, or —S—; Ar¹ represents a ring structure which forms a condensed ring with the oxygen-containing hetero-monocycle in the formula (3); R⁶ represents a monovalent substituent; n is 0 or 1; m is 0 or 1; r is an integer of 0 to 2; and “*” represents a site bonding to R² or R⁴,
wherein, when R² is bonded to the oxygen-containing hetero-monocycle in formula (2) or (3), R⁷ is not a single bond; when R⁴ is bonded to the oxygen-containing hetero-monocycle in formula (2) or (3), R⁴ is bonded to R³ via a carbon atom.

2: The radiation-sensitive composition according to claim 1, wherein the structural unit (I) is at least one species selected from the group consisting of a structural unit derived from a compound represented by formula (4) and a structural unit derived from a compound represented by formula (5): 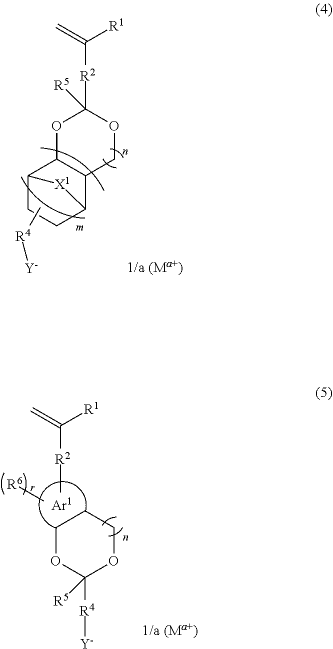
wherein R¹, R², R⁴, R⁵, R⁶, Y⁻, M^{a+}, X¹, Ar¹, a, n, m, and r each have the same meanings as defined in formulas (1) to (3).

3: The radiation-sensitive composition according to claim 1, wherein the radiation-sensitive composition, optionally, further comprises an additional polymer differing from the polymer comprising the structural unit (I), and at least one of the polymer comprising the structural unit (I) and the additional polymer comprises a structural unit (II) having an acid-releasable group.

4: The radiation-sensitive composition according to claim 1, wherein the polymer comprising the structural unit (I) further comprises a structural unit (III), the structural unit (III) comprising an aromatic ring and a hydroxy group bonded to the aromatic ring.

5: A resist pattern formation method, comprising:
forming a resist film on a substrate by applying the radiation-sensitive composition according to claim 1;
exposing the resist film to light; and
developing the resist film which has been exposed to the light.

6: A polymer comprising a structural unit represented by formula (1): 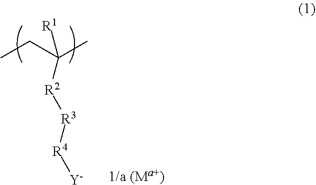
wherein R¹ represents a hydrogen atom, a fluorine atom, a methyl group, or a trifluoromethyl group; R² represents a single bond, a divalent hydrocarbon group, a divalent group F¹ which is formed by substituting any hydrogen atom of a divalent hydrocarbon group with a monovalent substituent, a divalent group F² including —O—, —CO—, —NH—, —COO—, or —CONH— which intervenes between a C—C bond of a divalent hydrocarbon group or the F¹, *¹—COO—R⁷—, or *¹—CONH—R⁷—; R⁷ represents a single bond, a divalent hydrocarbon group, a divalent group F³ which is formed by substituting any hydrogen atom of a divalent hydrocarbon group with a monovalent substituent, or a divalent group F⁴ including —O—, —CO—, —NH—, —COO—, or —CONH— which intervenes between a C—C bond of a divalent hydrocarbon group or the F³; “*¹” represents a site bonding to the carbon atom to which R¹ is bonded; R³ represents a divalent group represented by formula (2) or (3): R⁴ represents a divalent organic group: Y⁻ represents a monovalent anion which can generate a sulfonate group, an imidic acid group, or a methide acid group through exposure to light; M^{a+} represents an a-valent cation; and a is 1 or 2: 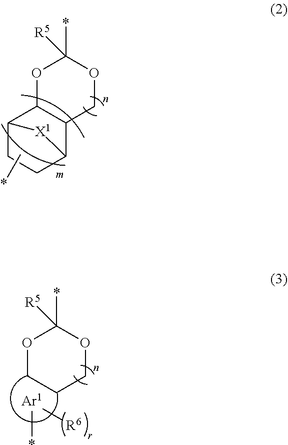
wherein R⁵ represents a hydrogen atom or a monovalent organic group; X¹ represents —CH₂—, —NH—, —O—, or —S—; Ar¹ represents a ring structure which forms a condensed ring with the oxygen-containing hetero-monocycle in the formula (3); R⁶ represents a monovalent substituent; n is 0 or 1; m is 0 or 1; r is an integer of 0 to 2; and “*” represents a site bonding to R² or R⁴,
wherein, when R² is bonded to the oxygen-containing hetero-monocycle in formula (2) or (3), when R⁴ is bonded to the oxygen-containing hetero-monocycle in formula (2) or (3), R⁴ is bonded to R³ via a carbon atom.

7: A compound represented by formula (4): 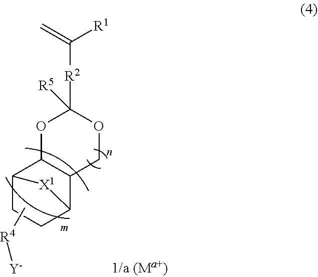
wherein R¹ represents a hydrogen atom, a fluorine atom, a methyl group, or a trifluoromethyl group; R² represents a single bond, a divalent hydrocarbon group, a divalent group F¹ which is formed by substituting any hydrogen atom of a divalent hydrocarbon group with a monovalent substituent, a divalent group F² including —O—, —CO—, —NH—, —COO—, or —CONH— which intervenes between a C—C bond of a divalent hydrocarbon group or the F¹, *¹—COO—R⁷—, or *¹—CONH—R⁷—; R⁷ represents a divalent hydrocarbon group, a divalent group F³ which is formed by substituting any hydrogen atom of a divalent hydrocarbon group with a monovalent substituent, or a divalent group F⁴ including —O—, —CO—, —NH—, —COO—, or —CONH— which intervenes between a C—C bond of a divalent hydrocarbon group or the F³; “*¹” represents a site bonding to the carbon atom to which R¹ is bonded; R⁴ represents a divalent organic group; R⁵ represents a hydrogen atom or a monovalent organic group; X¹ represents —CH₂—, —NH—, —O—, or —S—; Y⁻ represents a monovalent anion which can generate a sulfonate group, an imidic acid group, or a methide acid group through exposure to light; M^{a+} represents an a-valent cation; n is 0 or 1; m is 0 or 1; and a is 1 or 2.

8: A compound represented by formula (5): 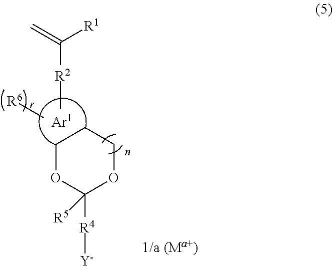
wherein R¹ represents a hydrogen atom, a fluorine atom, a methyl group, or a trifluoromethyl group; R² represents a single bond, a divalent hydrocarbon group, a divalent group F¹ which is formed by substituting any hydrogen atom of a divalent hydrocarbon group with a monovalent substituent, a divalent group F² including —O—, —CO—, —NH—, —COO—, or —CONH— which intervenes between a C—C bond of a divalent hydrocarbon group or the F¹, *¹—COO—R⁷—, or *¹—CONH—R⁷—; R⁷ represents a single bond, a divalent hydrocarbon group, a divalent group F³ which is formed by substituting any hydrogen atom of a divalent hydrocarbon group with a monovalent substituent, or a divalent group F⁴ including —O—, —CO—, —NH—, —COO—, or —CONH— which intervenes between a C—C bond of a divalent hydrocarbon group or the F³; “*¹” represents a site bonding to the carbon atom to which R¹ is bonded; Ar¹ represents a ring structure which forms a condensed ring with the oxygen-containing hetero-monocycle in formula (5); R⁴ represents a divalent organic group, wherein R⁴ is bonded to the oxygen-containing hetero-monocycle at a carbon atom; R⁵ represents a hydrogen atom or a monovalent organic group; R⁶ represents a monovalent substituent; n is 0 or 1; r is an integer of 0 to 2; Y⁻ represents a monovalent anion which can generate a sulfonate group, an imidic acid group, or a methide acid group through exposure to light; M^{a+} represents an a-valent cation; and a is 1 or 2.
